(19) United States
(12) Patent Application Publication
(10) Pub. No.: US 2012/0329993 A1
(43) Pub. Date: Dec. 27, 2012

(54) SOY WHEY PROTEIN COMPOSITIONS AND METHODS FOR RECOVERING SAME

(75) Inventors: Darrell Marsh, High Ridge, MO (US); Karsten Keller, Hockessin, DE (US); Charles S. Schasteen, University City, MO (US); Barry Tulk, Creve Coeur, MO (US); Marlene Mekel, High Ridge, MO (US); Jennifer Wu, ST. Charles, MO (US)

(73) Assignee: LEGAL DEPARTMENT, ST. LOUIS, MO (US)

(21) Appl. No.: 13/519,829

(22) PCT Filed: Dec. 30, 2010
(86) PCT No.: PCT/US10/62591

§ 371 Date: Jun. 28, 2012

Related U.S. Application Data
(60) Provisional application No. 61/291,312, filed on Dec. 30, 2009.

(51) Int. Cl. C07K 14/415; C12S 3/14

(52) U.S. Cl. 530/370; 435/272

(57) ABSTRACT

A process for recovering and isolating soy whey proteins and other components from whey processing streams is disclosed.

### CROSS REFERENCE TO RELATED APPLICATIONS

This application claims priority from U.S. Provisional Application Ser. No. 61/291,312 filed on Dec. 30, 2009, which is hereby incorporated by reference in its entirety.

### FIELD OF THE INVENTION

The present disclosure provides processes for the recovery of target proteins and other useful constituents from soy whey processing streams. Specifically, the present disclosure provides processes that utilize one or more membrane or chromatographic separation operations for isolating and removing soy proteins, including novel soy whey proteins and purified target proteins, as well as sugars, minerals, and other constituents to form a purified waste water stream.

### BACKGROUND OF THE INVENTION

The soybean, Glycine max, is a leguminous crop grown in many parts of the world. Soybeans are of great economic importance as a source of edible oil, high-protein foods, food ingredients, and stockfeed, as well as many industrial products.

Soy proteins are typically in one of three forms when consumed by humans. These include flour (grits), concentrates, and isolates. All three types are made from defatted soybean flakes. Flours and grits contain at least 50% protein and are prepared by milling the flakes. Soy protein concentrates contain 65 wt. % to 90 wt. % protein on a dry weight basis, with the major non-protein component being fiber. Concentrates are made by repeatedly washing the soy flakes with water, which may optionally contain low levels of food grade alcohols or buffers. The effluent from the repeated washings is discarded and the solid residue is dried, thereby producing the desired concentrate. The yield of concentrates from the starting material is approximately 60-70%.

Soy protein concentrate preparation generally results in two streams: soy isolate and a soy molasses stream, which may contain up to 55 wt. % soy protein. On a commercial scale, significant volumes of this molasses are generated that must be discarded. The total protein content may contain up to 15 wt. % of the total protein content of the soybeans from which they are derived. Thus, a significant fraction of soy protein is discarded during processes typically used for soy protein concentrate preparation.

Soy protein isolates are the most highly refined soy protein products commercially available, as well as the most expensive. However, as with soy protein concentrates, many of the valuable minerals, vitamins, isoflavones, and phytoestrogens are drawn off to form a waste stream along with the low-molecular weight sugars in making the isolates. Soy protein isolates contain a minimum of 90% protein on a dry weight basis and little or no soluble carbohydrates or fiber. Isolates are typically made by extracting defatted soy flakes or soy flour with a dilute alkali (pH<9) and centrifuging. The extract is adjusted to pH 4.5 with a food grade acid such as sulfuric, hydrochloric, phosphoric or acetic acid. At a pH of 4.5, the solubility of the proteins is at a minimum so they will precipitate out. The protein precipitate is then dried after being adjusted to a neutral pH or is dried without any pH adjustment to produce the soy protein isolate. The yield of the isolate is 30% to 50% of the original soy flour and 60% of the protein in the flour. This extremely low yield along with the many required processing steps contributes to the high costs involved in producing soy protein isolates.

Due at least in part to their relatively high protein content, soy protein isolates are desired for a variety of applications. In conventional soy protein isolate manufacture, the aqueous stream (i.e., soy whey stream) remaining after precipitation of the soy protein isolate fraction is typically discarded. On a commercial scale, considerable costs are incurred with the handling and disposing of this aqueous waste stream. For example, the soy whey stream is relatively dilute (e.g., less than about 5 wt. % solids, typically about 2 wt. % solids). Thus, on a commercial scale, significant volumes of the soy whey stream are generated that must be treated and/or discarded. In addition, it has been observed that the soy whey stream may contain a substantial proportion of the total protein content of the soybeans used in preparation of soy protein isolates. In fact, the soy whey stream may contain up to 45 wt. % of the total protein content of the soybeans from which soy protein isolates are derived. Thus, a significant fraction of soy protein is typically discarded during conventional soy protein isolate production.

Methods for recovering products from soy whey are known in the art. For example, a process for separating specific isoflavone fractions from soy whey and soy molasses feed streams is described in U.S. Pat. Nos. 6,033,714; 5,792,503; and 5,702,752. In another method, soy molasses (also referred to as soy solubles) is obtained when vacuum distillation removes the ethanol from an aqueous ethanol extract of defatted soy meal. The feed stream is heated to a temperature chosen according to the specific solubility of the desired isoflavone fraction. The stream is then passed through an ultrafiltration membrane, which allows isoflavone molecules below a maximum molecular weight to permeate. The permeate may then be concentrated using a reverse osmosis membrane. The concentrated stream is then put through a resin adsorption process using at least one liquid chromatography column to further separate the fractions.

Methods for the removal of oligosaccharides from soybean wastes are also known in the art. For example, Matsubara et al [Biosci. Biotech. Biochem. 60:421 (1996)] describe a method for recovering soybean oligosaccharides from steamed soybean wastewater using reverse osmosis and nanofiltration membranes. JP 07-082,287 teaches the recovery of oligosaccharides from soybean oligosaccharide syrup using solvent extraction. That method comprises adding an organic solvent to the aqueous solution containing the oligosaccharides, heating the mixture to give a homogeneous solution, cooling the solution to form two liquid layers, and separating and recovering the bottom layer.

Canadian Patent Applications 2,006,957 and 2,013,190 describe ion-exchange processes carried out in aqueous ethanol to recover small quantities of high value by-products from cereal grain processing waste. According to CA 2,013,190, an alcoholic extract from a cereal grain is processed through either an anionic and/or cationic ion-exchange column to obtain minor but economically valuable products.

Soy whey and soy molasses also contain a significant amount of protease inhibitors. Protease inhibitors are known to at least inhibit trypsin, chymotrypsin and potentially a variety of other key transmembrane proteases that regulate a range of key metabolic functions. Topical administration of protease inhibitors finds use in such conditions as atopic dermatitis, a common form of inflammation of the skin, which may be localized to a few patches or involve large portions of the body. The depigmenting activity of protease inhibitors and their capability to prevent ultraviolet-induced pigmentation have been demonstrated both in vitro and in vivo (See e.g., Paine et al., J. Invest. Dermatol., 116: 587-595 [2001]). Protease inhibitors have also been reported to facilitate wound healing. For example, secretory leukocyte protease inhibitor was demonstrated to reverse the tissue destruction and speed the wound healing process when topically applied. In addition, serine protease inhibitors can also help to reduce pain in lupus erythematosus patients (See e.g., U.S. Pat. No. 6,537,968). Naturally occurring protease inhibitors can be found in a variety of foods such as cereal grains (oats, barley, and maize), brussels sprouts, onion, beetroot, wheat, finger millet, and peanuts. One source of interest is the soybean.

Two broad classes of protease inhibitor superfamilies have been identified from soybean and other legumes with each class having several isoinhibitors. Kunitz-trypsin inhibitor (KTI) is major member of the first class whose members have approximately 170-200 amino acids, molecular weights between 20-25 kDa, and act principally against trypsin. Kunitz-trypsin proteinase inhibitors are mostly single chain polypeptides with 4 cysteines linked in two disulfide bridges, and with one reactive site located in a loop defined by disulfide bridge. The second class of inhibitors contains 60-85 amino acids, has a range in molecular weight of 6-10 kDa, has a higher number of disulfide bonds, is relatively heat-stable, and inhibits both trypsin and chymotrypsin at independent binding sites. Bowman-Birk inhibitor (BBI) is an example of this class. The average level of protease inhibitors present in soybeans is around 1.4 percent and 0.6 percent for KTI and BBI, respectively. Notably, these low levels make it impractical to isolate the natural protease inhibitor for clinical applications.

Preparing pure BBI, however, involves costly techniques. Moreover, because the average level of BBI present in soybeans is only around 0.6 wt. %, this low level makes it impractical and cost prohibitive to isolate the natural protease inhibitor for clinical applications. Purification methods currently used in the art vary. Some methods use affinity purification with immobilized trypsin or chymotrypsin. Immobilized trypsin will bind both BBI and Kunitz trypsin inhibitor (KTI) so a particularly pure BBI product is not isolated. Alternatively, a process involving use of immobilized chymotrypsin, while it does not bind KTI, has several problems, such as not being cost effective for scale-up and the possibility of chymotrypsin leaching from the resin following numerous uses and cleaning steps. Many older BBI purification methods use anion exchange chromatography, which technique can result in subfractionation of BBI isomers, In addition, it has been difficult with anion exchange chromatography to obtain a KTI-free BBI fraction without significant loss of BBI yield. Accordingly, all of the methods currently known for isolating BBI are problematic due to slow processing, low yield, low purity, and/or the need for a number of different steps which results in an increase of time and cost requirements.

Methods of purification which only utilize filtration are not effective as sole methods due to membrane fouling, incomplete and/or imperfect separation of non-protein components from BBI proteins, and ineffective separation of BBI proteins from other proteins. Methods of purification which only utilize chromatography are also not effective as sole methods due to binding capacity and overloading issues, incomplete and/or imperfect separation issues (e.g. separation of BBI from KTI), irreversible binding of protein to resin issues, resin lifetime issues, and it is relatively expensive compared to other techniques. Methods of purification which involve only ammonium sulfate precipitation are not effective as sole methods due to the possibility of irreversible precipitation of BBI proteins, potential loss of activity of BBI proteins, incomplete precipitation of BBI proteins (i.e. loss of yield), and the need to remove the ammonium sulfate from the final product, which adds an additional step and cost.

Despite the high proportion of the soy whey protein that is typically lost in the processing stream, recovery of the proteins has not generally been considered to be economically feasible. At least in part, the loss of these potentially valuable proteins has been heretofore deemed acceptable because of the relatively low concentrations of total protein in the whey, and the consequently large volumes of aqueous waste that must be processed for each unit of mass of protein recovered, which generates a large amount of pollution. Recovery attempts have also been deterred by the complex mixture of proteins and other components present in the soy whey, and the absence of commercial applications for crude mixtures of the protein solids. While soy whey has been known to contain certain bioactive proteins, the commercial value of these has been limited for lack of processes to recover them in high purity form.

Current methods known in the art for obtaining purified BBI proteins suffer from lower purity levels due to the contamination of the BBI with Kunitz Trypsin Inhibitor (KTI) proteins. Depending on the isolation method used, endotoxin levels can also be an issue. Current methods use whole soybean as the starting material, which is then defatted by various means. In contrast, the processes of the present invention use defatted soy white flake as the starting material. As a result, the prior art has not described a BBI product having high purity levels that is obtained from a soy protein source, without acid or alcohol extraction, or acetone precipitation. Thus, there is a need for methods and compositions suitable for the production of high purity BBI and variants.

Thus, there is a need in the art for a process that can be used to recover novel soy whey proteins, as well as other useful components, from a soy processing stream and thereby reduce pollution generated from the disposal of such waste on a large scale. Accordingly, the present invention describes novel methods for isolating various components in high purity from a soy processing stream. In addition, the methods of the present invention utilize fewer steps than the methods currently known in the art which resultingly reduces both time and cost requirements.

### SUMMARY OF THE INVENTION

The present disclosure relates to novel methods for purifying soy processing streams, and further encompasses novel methods for recovering soy whey proteins and other constituents therefrom. Specifically, the present disclosure provides processes that utilize one or more membrane or chromatographic separation operations for isolating and removing soy whey proteins, as well as sugars, minerals, and other constituents to form a purified waste water stream.

In another aspect, the present method results in the isolation and removal of one or more soy whey proteins from a soy processing stream, the soy processing stream comprising the soy whey proteins, one or more soy storage proteins, one or more sugars, and one or more minerals.

In a further aspect, the present method results in the isolation and removal of one or more sugars from a soy processing stream, the soy processing stream comprising the one or more sugars, one or more soy whey proteins, one or more soy storage proteins, and one or more minerals.

In yet a further aspect, the present method results in the isolation and removal of one or more minerals from a soy processing stream, the soy processing stream comprising the one or more minerals, one or more soy whey proteins, one or more soy storage proteins, and one or more sugars.

The present method relates to a process comprised of at least two sequential steps whereby various protein species are isolated as part of the treatment of a soy processing stream. Resultingly, the following proteins can be isolated by methods of the present invention: BBI proteins, KTI proteins, and combinations thereof. In addition to protein isolation, sugars and minerals can also be isolated and removed from the soy processing stream, to form purified waste water.

A sequence of steps, which are described below in further detail, are combined in varying order to comprise the overall process for recovering soy whey protein from a processing stream. Generally, the steps may be outlined as follows.

Step 0 is a whey protein pretreatment step which begins with the pretreatment of a feed stream. The feed stream is subjected to various processing aids and separation techniques, which results in the production of a stream comprised of soluble components in the aqueous phase of the stream and a stream comprised of insoluble large molecular weight proteins such as pre-treated soy whey, storage proteins, and combinations thereof.

Step 1 is a microbiology reduction step, which may begin with the stream obtained from Step 0 (e.g. pre-treated soy whey). The pre-treated soy whey is subjected to at least one separation technique to form a stream comprised of various components including but not limited to storage proteins, microorganisms, silicon, and combinations thereof, and a stream comprised of purified pre-treated soy whey.

Step 2 is a water and mineral removal step, which may begin with either the purified pre-treated soy whey stream obtained in Step 1 or the pre-treated soy whey stream obtained in Step 0 or demineralized pre-treated soy whey in Step 4. The pre-treated soy whey is subjected to at least one separation technique to form a stream comprised of purified pre-treated soy whey and a stream comprised of water, minerals, monovalent cations, and combinations thereof.

Step 3 is a mineral precipitation step, which may begin with either the purified pre-treated soy whey stream from Step 2 or the pre-treated soy whey stream from Step 0 or stream from Step 1. The pre-treated soy whey is subjected to at least one separation technique which results in a suspension of purified pre-treated soy whey and precipitated minerals.

Step 4 is a mineral removal step, which begins with the suspension of purified pre-treated soy whey and precipitated minerals from Step 3. The suspension and precipitation are subjected to at least one separation technique to form a stream comprised of de-mineralized pre-treated soy whey and a stream comprised of insoluble materials with protein mineral complexes.

Step 5 is a protein separation and concentration step, which begins with the purified pre-treated soy whey stream from Step 4 or the pre-treated soy whey from Steps 0, 1, or 2. The purified pre-treated soy whey is subjected to at least one separation technique to form a stream comprised of soy whey protein, BBI, KTI, storage proteins, other proteins and combinations thereof, and a stream comprised of peptides, soy oligosaccharides, minerals, and combinations thereof.

Step 6 is a protein washing a purification step, which can start with the protein stream from Step 4 or 5, or the pre-treated soy whey from Steps 0, 1, or 2. The proteins are subjected to at least one separation technique to form a stream comprised of soy whey protein, BBI, KTI, storage proteins, other proteins (e.g. lunasin, lectins, dehydrins, lipoxygenase) and combinations thereof and a stream comprised of peptides, soy oligosaccharides (e.g. sucrose, raffinose, stachyose, verbascose, monosaccharides), water, minerals, and combinations thereof.

Step 7 is a water removal step that can start with the stream from Step 5 and/or 6. The stream(s) are subjected to at least one separation technique to form a stream comprising peptides, soy oligosaccharides, water, minerals and combinations thereof and a stream comprising water, minerals, and combinations thereof.

Step 8 is a mineral removal step that can start with the stream from Step 5, 6, and/or stream of 7. The soy oligosaccharides are subjected to at least one separation technique to form a stream comprising de-mineralized soy oligosaccharides and a stream comprising minerals, water and combinations thereof.

Step 9 is a color removal step that can start with the soy oligosaccharides from Step 7, 8, 5 and/or 6. The soy oligosaccharides are subjected to at least one separation technique to form a stream comprising color compounds and a stream comprising soy oligosaccharides.

Step 10 is a soy oligosaccharide fractionation step that can start with the soy oligosaccharides from Step 9, 5, 6, 7, and/or 8. The soy oligosaccharides are subjected to at least one separation technique to form a stream comprising soy oligosaccharides such as sucrose, monosaccharides, and combinations thereof and a stream comprising soy oligosaccharides such as raffinose, stachyose, verbascose and combinations thereof.

Step 11 is a water removal step that can start with the soy oligosaccharides from Step 10, 9, 8, 7, 6, and/or 5. The soy oligosaccharides are subjected to at least one separation technique to form a stream comprising water and a stream comprising soy oligosaccharides.

Step 12 is an additional protein separation step that can start with the stream from Step 7, 5 and/or 6 (i.e. peptides, soy oligosaccharides, water and minerals). The stream is subjected to at least one separation technique to form a stream comprising soy oligosaccharides, water, minerals and combinations thereof (which can also be used as a starting material in Step 8) and a stream comprising peptides and other proteins.

Step 13 is a water removal step that can start with the peptides and other proteins from Step 12. The peptides and other proteins are subjected to at least one separation technique to form a stream comprising water and a stream comprising peptides and other proteins such as lunasin, lectins, dehydrins, lipoxygenase, and combinations thereof.

Step 14 is a protein fractionation step that can start with the protein stream from Steps 5 and/or 6. The proteins are subjected to at least one separation technique to form a stream comprising storage proteins and a stream comprising soy whey protein, BBI, KTI, and other proteins such as lunasin, lectins, dehydrins, lipoxygenase and combinations thereof. The characteristics of these proteins are set forth in FIG. 1.

Step 15 is a water removal step that can start with the soy whey protein, BBI, KTI and other proteins from Steps 5, 6 and/or 14. The proteins are subjected to at least one separation technique to form a stream comprising water and a stream comprising soy whey protein, BBI, KTI and other proteins.

Step 16 is a heat treatment and flash cooling step that can start with the soy whey protein, BBI, KTI and other proteins from Steps 5, 6, 14, and or 15. The proteins are subjected to ultra high temperatures to form soy whey protein.

Step 17 is a drying step that can start with the soy whey protein, BBI, KTI and other proteins from Steps 5, 6, 14, 15, and/or 16. The proteins are dried to form a stream comprising water and a stream comprising soy whey protein.

### BRIEF DESCRIPTION OF THE DRAWINGS

FIG. 1 is a chart setting forth the proteins found in whey streams and their characteristics.

FIG. 2 graphically depicts the solubility of the soy whey proteins over a pH range of 3-7 as compared to that of soy protein isolates.

FIG. 3 graphically depicts the rheological properties of the soy whey proteins compared to soy protein isolate.

FIG. 4A is a schematic flow sheet depicting Steps 0 through 4 in a process for recovery of a purified soy whey protein from processing stream.

FIG. 4B is a schematic flow sheet depicting Steps 5, 6, 14, 15, 16, and 17 in a process for recovery of a purified soy whey protein from processing stream.

FIG. 4C is a schematic flow sheet depicting Steps 7 through 13 in a process for recovery of a purified soy whey protein from processing stream.

FIG. 5 graphically illustrates the breakthrough curve when loading soy whey at 10, 15, 20 and 30 mL/min (5.7, 8.5, 11.3, 17.0 cm/min linear flow rate, respectively) through a SP Gibco cation exchange resin bed plotted against empty column volumes loaded.

FIG. 6 graphically illustrates protein adsorption on SP Gibco cation exchange resin when passing soy whey at 10, 15, 20 and 30 mL/min (5.7, 8.5, 11.3, 17.0 cm/min linear flow rate, respectively) plotted against empty column volumes loaded.

FIG. 7 graphically illustrates the breakthrough curve when loading soy whey at 15 mL/min and soy whey concentrated by a factor of 3 and 5 through SP Gibco cation exchange resin bed plotted against empty column volumes loaded.

FIG. 8 graphically illustrates protein adsorption on SP Gibco cation exchange resin when passing soy whey and soy whey concentrated by a factor of 3 and 5 at 15 mL/min through SP Gibco cation exchange resin bed plotted against empty column volumes loaded.

FIG. 9 graphically depicts equilibrium protein adsorption on SP Gibco cation exchange resin when passing soy whey and soy whey concentrated by a factor of 3 and 5 at 15 mL/min through SP Gibco cation exchange resin bed plotted against equilibrium protein concentration in the flow through.

FIG. 10 graphically illustrates the elution profiles of soy whey proteins desorbed with varying linear velocities over time.

FIG. 11 graphically illustrates the elution profiles of soy whey proteins desorbed with varying linear velocities with column volumes.

FIG. 12 depicts a sodium dodecyl sulfate polyacrylamide gel electrophoresis (SDS-PAGE) analysis of Mimo6ME fractions.

FIG. 13 depicts a SDS-PAGE analysis of Mimo4SE fractions.

FIG. 14 depicts a SDS-PAGE analysis of Mimo6HE fractions.

FIG. 15 depicts a SDS-PAGE analysis of Mimo6ZE fractions.

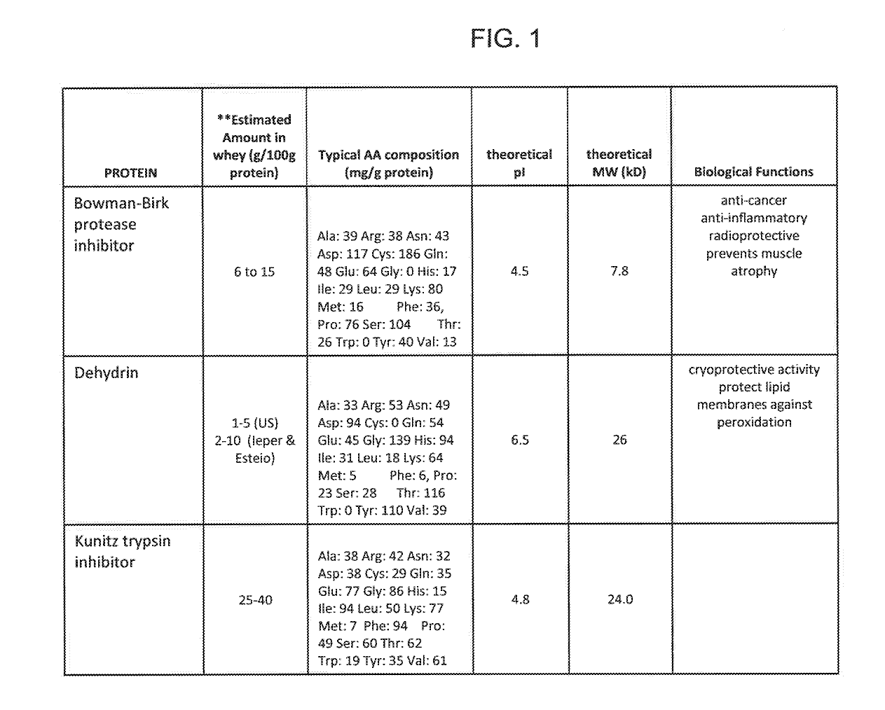

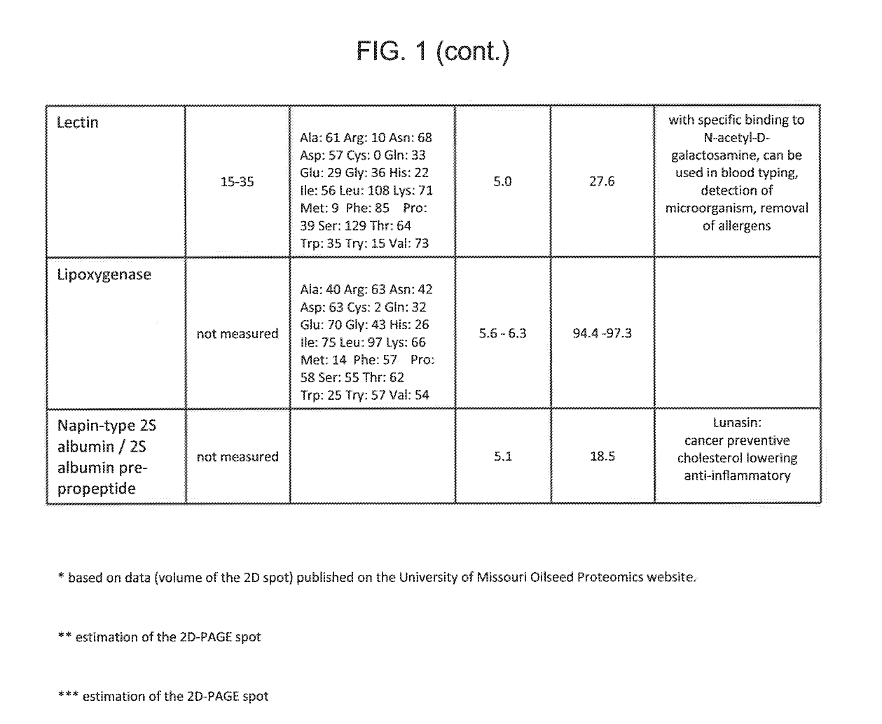

### DETAILED DESCRIPTION OF THE PREFERRED ASPECTS

Described herein are novel processes for recovering highly purified target proteins and other products from a variety of leguminous plant processing streams (including soy whey streams and soy molasses streams) generated in the manufacture of soy protein isolates. Generally, the processes of the present disclosure comprise one or more operations (e.g. membrane separation operations) selected and designed to provide recovery of the desired proteins or other products, or separation of various components of the soy whey stream, or both. Recovery of soy whey proteins (e.g. Bowman-Birk inhibitor (BBI) and Kunitz trypsin inhibitor (KTI) proteins) and one or more other components of the soy whey stream (e.g. various sugars, including oligosaccharides) may utilize a plurality of separation techniques, (e.g. membrane, chromatographic, centrifugation, or filtration). The specific separation technique will depend upon the desired component to be recovered by separating it from other components of the processing stream.

For example, a purified fraction is typically prepared by removal of one or more impurities (e.g. microorganisms or minerals), followed by removal of additional impurities including one or more soy storage proteins (i.e. glycinin and β-conglycinin), followed by removal of one or more soy whey proteins (including, for example, KTI and other non-BBI proteins or peptides), and/or followed by removal of one or more additional impurities including sugars from the soy whey. Recovery of various target components in high purity form is improved by removal of other major components of the whey stream (e.g. storage proteins, minerals, and sugars) that detract from purity by diluents, while likewise improving purity by purifying the protein fraction through removal of components that are antagonists to the proteins and/or have deleterious effects (e.g. endotoxins). Removal of the various components of the soy whey typically comprises concentration of the soy whey prior to and/or during removal of the components of the soy whey. The methods of the present invention also will reduce pollution generated from processing large quantities of aqueous waste.

Removal of storage proteins, sugars, minerals, and impurities yields fractions that are enriched in the individual, targeted proteins and free of impurities that may be antagonists or toxins, or may otherwise have a deleterious effect. For example, typically a soy storage protein-enriched fraction may be recovered, along with a fraction enriched in one or more soy whey proteins. A fraction enriched in one more sugars (e.g. oligosaccharides and/or polysaccharides) is also typically prepared. Thus, the present methods provide a fraction that is suitable as a substrate for recovery of individual, targeted proteins, and also provide other fractions that can be used as substrates for economical recovery of other useful products from aqueous soy whey. For example, removal of sugars and/or minerals from the soy whey stream produces a useful fraction from which the sugars can be further separated, thus yielding additional useful fractions: a concentrated sugar and a mineral fraction (that may include citric acid), and a relatively pure aqueous fraction that may be disposed of with minimal, if any, treatment or recycled as process water. Process water thus produced may be especially useful in practicing the present methods. Thus, a further advantage of the present methods may be reduced process water requirements as compared to conventional isolate preparation processes.

Methods of the present disclosure provide advantages over conventional methods for manufacture of soy protein isolates and concentrates in at least two ways. As noted, conventional methods for manufacturing soy protein materials typically dispose of the soy whey stream (e.g. aqueous soy whey or soy molasses). Thus, the products recovered by the methods of the present disclosure represent an additional product, and a revenue source not currently realized in connection with conventional soy protein isolate and soy protein concentrate manufacture. Furthermore, treatment of the soy whey stream or soy molasses to recover products preferably reduces the costs associated with treatment and disposal of the soy whey stream or soy molasses. For example, as detailed elsewhere herein, various methods of the present invention provide a relatively pure soy processing stream that may be readily utilized in various other processes or disposed of with minimal, if any, treatment, thereby reducing the environmental impact of the process. Certain costs exist in association with the methods of the present disclosure, but the benefits of the additional product(s) isolated and minimization of waste disposal compensate for any added costs.

### A. Soy Whey Proteins

The soy whey proteins recovered in accordance with the processes of the present disclosure represent a significant advance in the art over other soy proteins and isolates. As noted herein, the soy whey proteins of the present disclosure, which are recovered from a processing stream, possess unique characteristics as compared to other soy proteins found in the art.

Soy protein isolates are typically precipitated from an aqueous extract of defatted soy flakes or soy flour at the isoelectric point of soy storage proteins (e.g. a pH of about 4.5). Thus, soy protein isolates generally include proteins that are not soluble in acidic liquid media. Similarly, the proteins of soy protein concentrates, the second-most refined soy protein material, are likewise generally not soluble in acidic liquid media. However, soy whey proteins recovered by the processes of the present disclosure differ in that they are generally acid-soluble, meaning they are soluble in acidic liquid media.

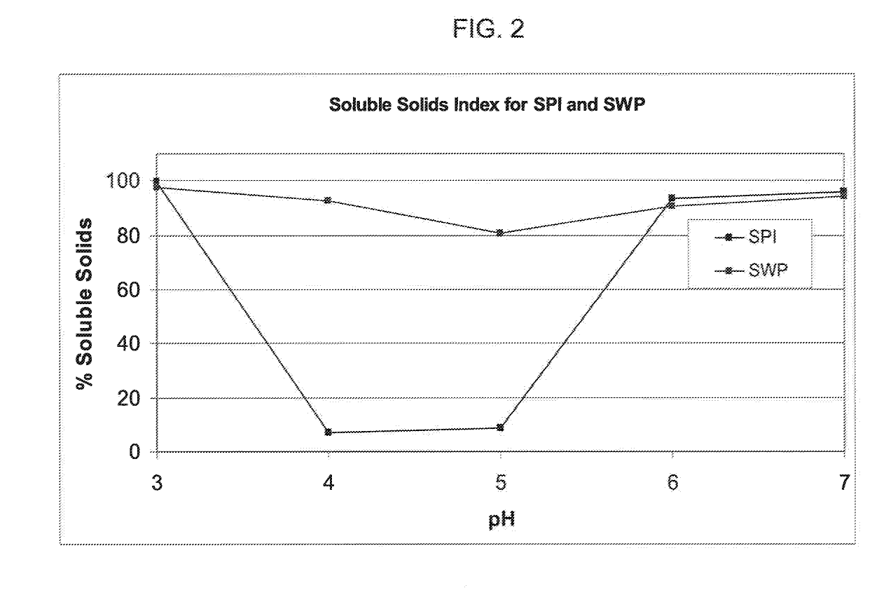

The present disclosure provides soy whey protein compositions derived from an aqueous soy whey that exhibit advantageous characteristics over soy proteins found in the prior art. For example, the soy whey proteins isolated according to the methods of the present invention possess high solubility (i.e. SSI % greater than 80) across a relatively wide pH range of the aqueous (typically acidic) medium (e.g. an aqueous medium having a pH of from about 2 to about 10, from about 2 to about 7, or from about 2 to about 6) at ambient conditions (e.g. a temperature of about 25° C.). As shown in Table 1 and graphically illustrated in FIG. 2, the solubility of the soy whey proteins isolated in accordance with the methods of the present disclosure, at all pH values tested, was at least 80%, and in all but one instance (i.e. pH 4) was at least about 90%. These findings were compared with soy protein isolate, which was shown to display poor solubility characteristics at the same acid pH values. This unique characteristic enables the soy whey proteins of the present invention to be used in applications having acidic pH levels, which represents a significant advantage over soy isolate.

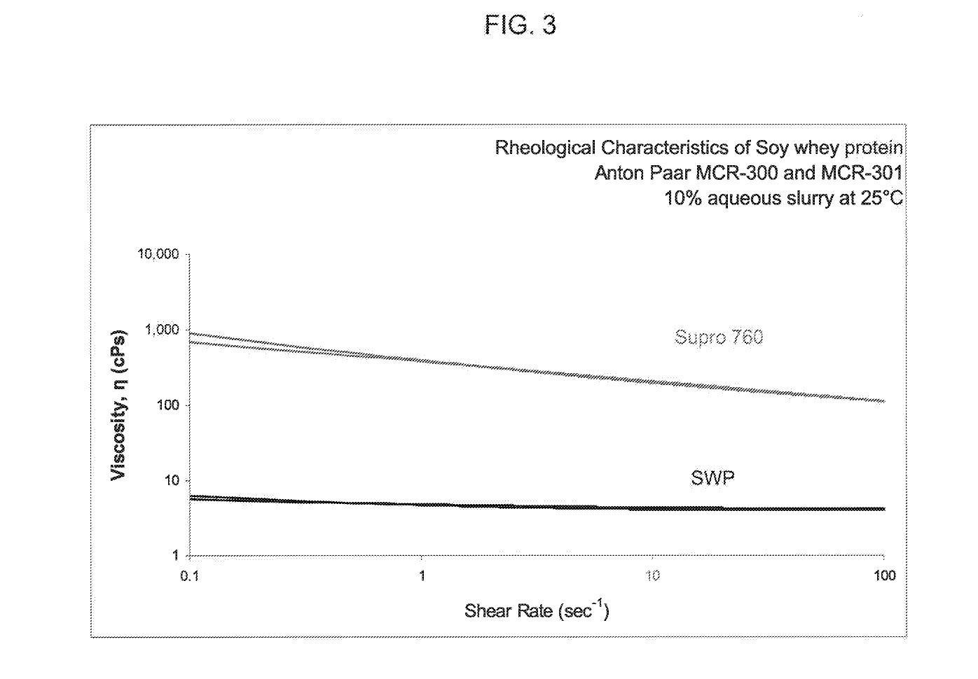

In addition to solubility, the soy whey proteins of the present disclosure also possess much lower viscosity than other soy whey proteins. As shown in Table 1 and as graphically illustrated in FIG. 3, the soy whey proteins of the present invention displayed viscoelastic properties (i.e. rheological properties) more similar to that of water than shown by soy protein isolate. The viscosity of water is about 1 centipoise (cP) at 20° C. The soy whey proteins of the present disclosure were found to exhibit viscosity within the range of from about 2.0 to 10.0 cP, and preferably from about 3.6 to 7.5 cP. This low viscosity, in addition to its high solubility at acidic pH levels, makes the soy whey protein of the present disclosure available and better suited for use in certain applications that regularly involve the use of other proteins (e.g., in acidic beverages), because it has much better flow characteristics than that of soy isolate.

TABLE 1
Solubility and Viscoelastic Properties of Soy Whey Compared to
Other Soy Proteins

| | SWP | Supro 500E |
|---|---|---|
| SSI %, pH 3.0 | 99 | 100 |
| SSI %, pH 4.0 | 82.3 | 7 |
| SSI %, pH 5.0 | 89.4 | 9 |
| SSI %, pH 6.0 | 99.3 | 94 |
| SSI %, pH 7.0 | 99.4 | 96 |
| viscosity, cPs | 4.3 | 385 |

### B. Aqueous Whey Streams

Aqueous whey streams and molasses streams, which are types of soy processing streams, are generated from the process of refining a whole legume or oilseed. The whole legume or oilseed may be derived from a variety of suitable plants. By way of non-limiting example, suitable plants include leguminous plants, including for example, soybeans, corn, peas, canola, sunflowers, sorghum, rice, amaranth, potato, tapioca, arrowroot, canna, lupin, rape, wheat, oats, rye, barley, and mixtures thereof. In one embodiment, the leguminous plant is soybean and the aqueous whey stream generated from the process of refining the soybean is an aqueous soy whey stream.

Aqueous soy whey streams generated in the manufacture of soy protein isolates are generally relatively dilute and are typically discarded as waste. More particularly, the aqueous soy whey stream typically has a total solids content of less than about 10 wt. %, typically less than about 7.5 wt. % and, still more typically, less than about 5 wt. %. For example, in various aspects, the solids content of the aqueous soy whey stream is from about 0.5 to about 10 wt. %, from about 1 wt. % to about 4 wt. %, or from about 1 to about 3 wt. % (e.g. about 2 wt. %). Thus, during commercial soy protein isolate production, a significant volume of waste water that must be treated or disposed is generated.

Soy whey streams typically contain a significant portion of the initial soy protein content of the starting material soybeans. As used herein the term “soy protein” generally refers to any and all of the proteins native to soybeans. Naturally occurring soy proteins are generally globular proteins having a hydrophobic core surrounded by a hydrophilic shell. Numerous soy proteins have been identified including, for example, storage proteins such as glycinin and β-conglycinin. Soy proteins likewise include protease inhibitors, such as the above-noted BBI and KTI proteins. Soy proteins also include hemagglutinins such as lectin, lipoxygenases, β-amylase, and lunasin. It is to be noted that the soy plant may be transformed to produce other proteins not normally expressed by soy plants. It is to be understood that reference herein to “soy proteins” likewise contemplates proteins thus produced.

On a dry weight basis, soy proteins constitute at least about 10 wt. %, at least about 15 wt. %, or at least about 20 wt. % of the soy whey stream (dry weight basis). Typically, soy proteins constitute from about 10 to about 40 wt. %, or from about 20 to about 30 wt. % of the soy whey stream (dry weight basis). Soy protein isolates typically contain a significant portion of the storage proteins of the soybean. However, the soy whey stream remaining after isolate precipitation likewise contains one or more soy storage proteins.

In addition to the various soy proteins, the aqueous soy whey stream likewise comprises one or more carbohydrates (i.e. sugars). Generally, sugars constitute at least about 25%, at least about 35%, or at least about 45% by weight of the soy whey stream (dry weight basis). Typically, sugars constitute from about 25% to about 75%, more typically from about 35% to about 65% and, still more typically, from about 40% to about 60% by weight of the soy whey stream (dry weight basis).

The sugars of the soy whey stream generally include one or more monosaccharides, and/or one or more oligosaccharides or polysaccharides. For example, in various aspects, the soy whey stream comprises monosaccharides selected from the group consisting of glucose, fructose, and combinations thereof. Typically, monosaccharides constitute from about 0.5% to about 10 wt. % and, more typically from about 1% to about 5 wt. % of the soy whey stream (dry weight basis). Further in accordance with these and various other aspects, the soy whey stream comprises oligosaccharides selected from the group consisting of sucrose, raffinose, stachyose, and combinations thereof. Typically, oligosaccharides constitute from about 30% to about 60% and, more typically, from about 40% to about 50% by weight of the soy whey stream (dry weight basis).

The aqueous soy whey stream also typically comprises an ash fraction that includes a variety of components including, for example, various minerals, isoflavones, phytic acid, citric acid, saponins, and vitamins. Minerals typically present in the soy whey stream include sodium, potassium, calcium, phosphorus, magnesium, chloride, iron, manganese, zinc, copper, and combinations thereof. Vitamins present in the soy whey stream include, for example, thiamine and riboflavin. Regardless of its precise composition, the ash fraction typically constitutes from about 5% to about 30% and, more typically, from about 10% to about 25% by weight of the soy whey stream (dry weight basis).

The aqueous soy whey stream also typically comprises a fat fraction that generally constitutes from about 0.1% to about 5% by weight of the soy whey stream (dry weight basis). In certain aspects of the invention, the fat content is measured by acid hydrolysis and is about 3% by weight of the soy whey stream (dry weight basis).

In addition to the above components, the aqueous soy whey stream also typically comprises one or more microorganisms including, for example, various bacteria, molds, and yeasts. The proportions of these components typically vary from about 100 to about 1×10⁹ colony forming units (CFU) per milliliter. As detailed elsewhere herein, in various aspects, the aqueous soy whey stream is treated to remove these component(s) prior to protein recovery and/or isolation.

As noted, conventional production of soy protein isolates typically includes disposal of the aqueous soy whey stream remaining following isolation of the soy protein isolate. In accordance with the present disclosure, recovery of one or more proteins and various other components (e.g. sugars and minerals) results in a relatively pure aqueous whey stream. Conventional soy whey streams from which the protein and one or more components have not been removed generally require treatment prior to disposal and/or reuse. In accordance with various aspects of the present disclosure the aqueous whey stream may be disposed of or utilized as process water with minimal, if any, treatment. For example, the aqueous whey stream may be used in one or more filtration (e.g. diafiltration) operations of the present disclosure.

In addition to recovery of BBI proteins from aqueous soy whey streams generated in the manufacture of soy protein isolates, it is to be understood that the processes described herein are likewise suitable for recovery of one or more components of soy molasses streams generated in the manufacture of a soy protein concentrate, as soy molasses streams are an additional type of soy processing stream.

### C. General Description of Process for Soy Whey Protein Recovery

The following is a general description of the various steps that make up the overall process. A key to the process is to start with the whey protein pretreatment step, which uniquely changes the soy whey and protein properties. From there, the other steps may be performed using the raw material sources as listed in each step, as will be shown in the discussion of the various embodiments to follow.

It is understood by those skilled in the art of separation technology that there can be residual components in each stream since separation is never 100%. Further, one skilled in the art realizes that separation technology can vary depending on the starting raw material.

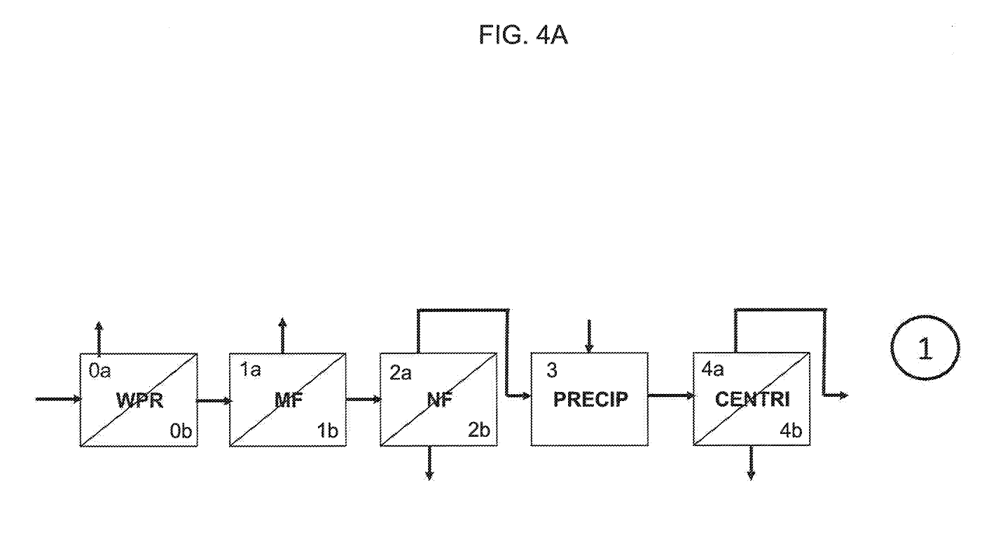

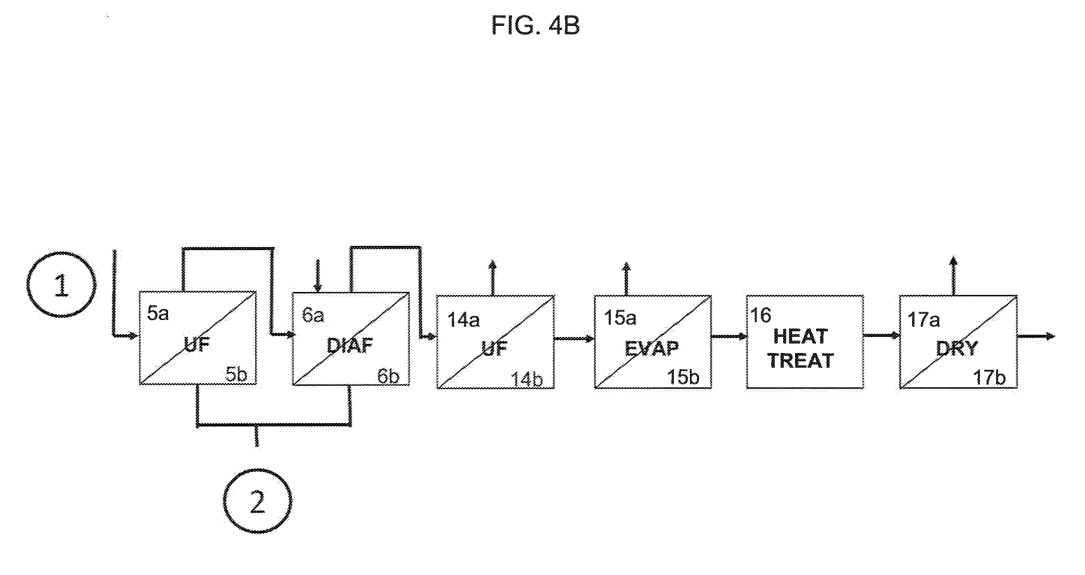

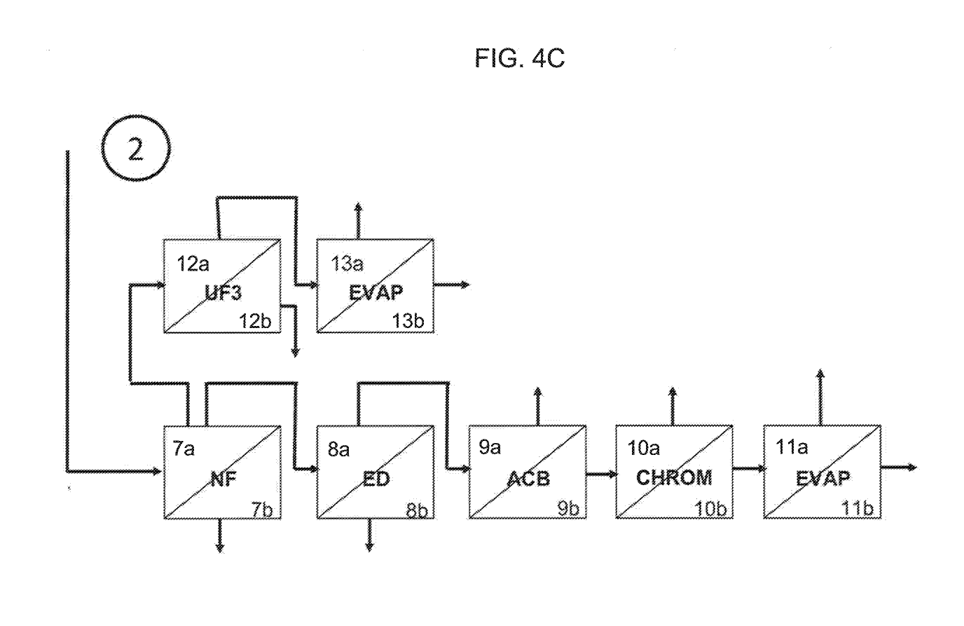

Step 0 (as shown in FIG. 4A)—Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 1 (as shown in FIG. 4A)—Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, or between about 3.5 and about 5.5, or about 5.3. The temperature can be between about 5° C. and about 90° C., or between about 25° C. and about 75° C., or about 50° C. Products from step 1 include but are not limited to storage proteins, microorganisms, silicon, and combinations thereof in stream 1a and purified pre-treated soy whey in stream 1b.

Step 2 (as shown in FIG. 4A)—A water and mineral removal can start with the purified pre-treated soy whey from stream 1b or 4a, or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 3 (as shown in FIG. 4A)—the mineral precipitation step can start with purified pre-treated soy whey from stream 2a or pretreated soy whey from streams 0a or 1b. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (as shown in FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (as shown in FIG. 4B)—the protein separation and concentration step can start with purified pre-treated whey from stream 4a or the whey from streams 0a, 1b, or 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (as shown in FIG. 4B)—the protein washing and purification step can start with soy whey protein, BBI, KTI, storage proteins, other proteins or purified pre-treated whey from stream 4a or 5a, or whey from streams 0a, 1b, or 2a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 7 (as shown in FIG. 4C)—a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., or between about 25° C. and about 75° C., or about 50° C. Products from stream 7a include but are not limited to, peptides, soy oligosaccharides, water, minerals, and combinations thereof. Products from stream 7b include but are not limited to, water, minerals, and combinations thereof.

Step 8 (as shown in FIG. 4C)—a mineral removal step can start with peptides, soy oligosaccharides, water, minerals, and combinations thereof from streams 5b, 6b, 7a, and/or 12b. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, or between about 6.0 and about 9.0, or about 7.0. The temperature can be between about 5° C. and about 90° C., or between about 25° C. and about 50° C., or about 40° C. Products from stream 8a include but are not limited to, de-mineralized soy oligosaccharides with conductivity between about 10 milli Siemens/centimeter (mS/cm) and about 0.5 mS/cm, or about 2 mS/cm. Products from stream 8b include but are not limited to, minerals, water, and combinations thereof.

Step 9 (as shown in FIG. 4C)—a color removal step can start with de-mineralized soy oligosaccharides from streams 8a, 5b, 6b, 12b, and/or 7a). It utilizes an active carbon bed. Process variables and alternatives in this step include but are not limited to, ion exchange. Processing aids that can be used in this color removal step include but are not limited to, active carbon, ion exchange resins, and combinations thereof. The temperature can be between about 5° C. and about 90° C., or about 40° C. Products from stream 9a include but are not limited to, color compounds. Stream 9b is a decolored solution. Products from stream 9b include but are not limited to, soy oligosaccharides, and combinations thereof.

Step 10 (as shown in FIG. 4C)—a soy oligosaccharide fractionation step can start with soy oligosaccharides, and combinations thereof from streams 9b, 5b, 6b, 7a, and/or 8a. It includes a chromatography step. Process variables and alternatives in this step include but are not limited to, chromatography, nanofiltration, and combinations thereof. Processing aids that can be used in this soy oligosaccharide fractionation step include but are not limited to acid or base to adjust the pH as one skilled in the art would know, based on the resin used. Products from stream 10a include but are not limited to, soy oligosaccharides. Products from stream 10b include but are not limited to soy oligosaccharides.

Step 11 (as shown in FIG. 4C)—a water removal step can start with soy oligosaccharides such as, raffinose, stachyose, verbascose, and combinations thereof from streams 9b, 5b, 6b, 7a, 8a, and/or 10b. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., or about 60° C. Products from stream 11a include but are not limited to, water. Products from stream 11b include but are not limited to, soy oligosaccharides.

Step 12 (as shown in FIG. 4C)—an additional protein separation from soy oligosaccharides step can start with peptides, soy oligosaccharides, water, minerals, and combinations thereof from stream 7a, 5b, and/or 6b. It includes an ultrafiltration step. Process variables and alternatives in this step include but are not limited to, crossflow membrane filtration, ultrafiltration with pore sizes between about 50 kDa and about 1 kDa, and combinations thereof. Crossflow membrane filtration includes but is not limited to: spiral-wound, plate and frame, hollow fiber, ceramic, dynamic or rotating disk, nanofiber, and combinations thereof. Processing aids that can be used in this protein separation from sugars step include but are not limited to, acids, bases, protease, phytase, and combinations thereof. The pH of step 12 can be between about 2.0 and about 12.0, or about 7.0. The temperature can be between about 5° C. and about 90° C., or between about 25° C. and about 75° C., or about 50° C. Products from stream 12b include but are not limited to, soy oligosaccharides, water, minerals, and combinations thereof. Products from stream 12a include but are not limited to, peptides, other proteins, and combinations thereof.

Step 13 (as shown in FIG. 4C)—a water removal step can start with, peptides, and other proteins from stream 12a. It includes an evaporation step. Process variables and alternatives in this step include but are not limited to, reverse osmosis, nanofiltration, spray drying and combinations thereof. Products from stream 13a include but are not limited to, water. Products from stream 13b include but are not limited to, peptides, other proteins, and combinations thereof.

Step 14 (as shown in FIG. 4B)—a protein fractionation step may be done by starting with soy whey protein, BBI, KTI, storage proteins, other proteins, and combinations thereof from streams 6a and/or 5a. It includes an ultrafiltration (with pore sizes from 300 kDa to 10 kDa) step. Process variables and alternatives in this step include but are not limited to, crossflow membrane filtration, ultrafiltration, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 14 can be between about 2.0 and about 12.0, or between about 6.0 and about 9.0, or about 7.0. The temperature can be between about 5° C. and about 90° C., or between about 25° C. and about 75° C., or about 50° C. Products from stream 14a include but are not limited to, storage proteins. Products from stream 14b include but are not limited to, soy whey protein, BBI, KTI, other proteins, and combinations thereof.

Step 15 (as shown in FIG. 4B)—a water removal step can start with soy whey protein, BBI, KTI and, other proteins from streams 6a, 5a, and/or 14b. It includes an evaporation step. Process variables and alternatives in this step include but are not limited to, evaporation, nanofiltration, reverse osmosis, and combinations thereof. Products from stream 15a include but are not limited to, water. Stream 15b products include but are not limited to soy whey protein, BBI, KTI, other proteins, and combinations thereof.

Step 16 (as shown in FIG. 4B)—a heat treatment and flash cooling step can start with soy whey protein, BBI, KTI and, other proteins from streams 6a, 5a, 14b, and/or 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Step 17 (as shown in FIG. 4B)—a drying step can start with soy whey protein, BBI, KTI, and other proteins from streams 6a, 5a, 14b, 15b, and/or 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI, other proteins, and combinations thereof.

The soy whey protein products of the current application include raw whey, a soy whey protein precursor after the ultrafiltration step of Step 17, a dry soy whey protein that can be dried by any means known in the art, and combinations thereof. All of these products can be used as is as soy whey protein or can be further processed to purify specific components of interest, such as, but not limited to BBI, KTI, and combinations thereof.

The soy whey protein products of the current application can have at least about 20 wt. % dry basis protein, at least about 60 wt. % dry basis protein, at least about 75 wt. % dry basis protein, at least about 80 wt. % dry basis protein, at least about 85 wt. % dry basis protein, at least about 90 wt. % dry basis protein, or at least about 95 wt. % dry basis protein.

### D. Preferred Embodiments of the Process for the Recovery of Soy Whey Protein

Embodiment 1 starts with Step 0 (See FIG. 4A) as follows: Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof. Next

Step 5 (See FIG. 4B) is done. Thus, the protein separation and concentration step in this embodiment starts with the whey from stream 0a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Embodiment 2—starts with Step 0 (See FIG. 4A) as follows: Whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Next Step 5 (See FIG. 4B) is done. Thus, the protein separation and concentration step in this embodiment starts with the whey from stream 0a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Finally Step 6 (See FIG. 4B), the protein washing and purification step starts with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Embodiment 3 starts with Step 0 (See FIG. 4A) which is a whey protein pretreatment that can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with purified pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Finally, Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Embodiment 4 starts with Step 0 (See FIG. 4A) whey protein pretreatment that can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with purified pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B)—the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Embodiment 5 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B)—the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 16 (See FIG. 4B) a heat treatment and flash cooling step can start with soy whey protein, BBI, KTI and, other proteins from streams 6a. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B)—a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 6 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 15 (See FIG. 4B) a water removal step can start with soy whey protein, BBI, KTI and, other proteins from stream 6a. It includes an evaporation step. Process variables and alternatives in this step include but are not limited to, evaporation, nanofiltration, reverse osmosis, and combinations thereof. Products from stream 15a include but are not limited to, water. Stream 15b products include but are not limited to soy whey protein, BBI, KTI and, other proteins.

Step 16 (See FIG. 4B) a heat treatment and flash cooling step can start with soy whey protein, BBI, KTI and, other proteins from stream 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B)—a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 7 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Finally, Step 5 (See FIG. 4B) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Embodiment 8 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 5 (See FIG. 48) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Embodiment 9 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Embodiment 10 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Embodiment 11 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 2 (See FIG. 4A) a water and mineral removal can start with the pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B)—the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 16 (See FIG. 4B) a heat treatment and flash cooling step can start with soy whey protein, BBI, KTI and, other proteins from stream 6a. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B)—a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 12 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 2 (See FIG. 4A) a water and mineral removal can start with the purified pre-treated soy whey from stream 1b or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 15 (See FIG. 4B) a water removal step can start with soy whey protein, BBI, KTI and, other proteins from stream 6a. It includes an evaporation step. Process variables and alternatives in this step include but are not limited to, evaporation, nanofiltration, reverse osmosis, and combinations thereof. Products from stream 15a include but are not limited to, water. Stream 15b products include but are not limited to soy whey protein, BBI, KTI and, other proteins.

Step 16 (See FIG. 4B) a heat treatment and flash cooling step can start with soy whey protein, BBI, KTI and, other proteins from stream 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 13 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with pre-treated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 2 (See FIG. 4A) a water and mineral removal can start with the purified pre-treated soy whey from stream 1b or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Finally, Step 5 (See FIG. 4B) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Embodiment 14 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with pretreated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 2 (See FIG. 4A) a water and mineral removal can start with the purified pre-treated soy whey from stream 4a. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 5 (See FIG. 4B) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Finally, Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Embodiment 15 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with pretreated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 2 (See FIG. 4A) a water and mineral removal can start with the purified pre-treated soy whey from stream 1b or pre-treated soy whey from stream 0b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 5 (See FIG. 4B) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 16 (See FIG. 4B) a heat treatment and flash cooling step can start with soy whey protein, BBI, KTI and, other proteins from stream 6a. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 16 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 3 (See FIG. 4A) the mineral precipitation step can start with pretreated soy whey from stream 0a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 2 (See FIG. 4A) a water and mineral removal can start with the purified pre-treated soy whey from stream 4a. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 5 (See FIG. 4B) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 15 (See FIG. 4B) a water removal step can start with soy whey protein, BBI, KTI and, other proteins from stream 6a. It includes an evaporation step. Process variables and alternatives in this step include but are not limited to, evaporation, nanofiltration, reverse osmosis, and combinations thereof. Products from stream 15a include but are not limited to, water. Stream 15b products include but are not limited to soy whey protein, BBI, KTI and, other proteins.

Step 16 (See FIG. 4B) a heat treatment and flash cooling step can start with soy whey protein, BBI, KTI and, other proteins from stream 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B) a drying step can start with soy whey protein BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 17 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 1 (See FIG. 4A) Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to: spiral-wound, plate and frame, hollow fiber, ceramic, dynamic or rotating disk, nanofiber, and combinations thereof. The pH of step 1 can be between about 2.0 and about 12.0, or between about 3.5 and about 5.5, or about 5.3. The temperature can be between about 5° C. and about 90° C., or between about 25° C. and about 75° C., or about 50° C. Products from step 1 include but are not limited to storage proteins, microorganisms, silicon, and combinations thereof in stream 1a and purified pre-treated soy whey in stream 1b.

Step 3 (See FIG. 4A) the mineral precipitation step can start with pretreated soy whey from stream 1b. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A) the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 2 (See FIG. 4A)—A water and mineral removal can start with the purified pre-treated soy whey from stream 4a. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C., or about 50° C. Products from this water removal step include but are not limited to purified pre-treated soy whey in stream 2a and water, some minerals, monovalent cations and combinations thereof in stream 2b.

Step 5 (See FIG. 4B) the protein separation and concentration step can start with the whey from stream 2a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 15 (See FIG. 4B) a water removal step can start with soy whey protein, BBI, KTI and, other proteins from stream 6a. It includes an evaporation step. Process variables and alternatives in this step include but are not limited to, evaporation, nanofiltration, reverse osmosis, and combinations thereof. Products from stream 15a include but are not limited to, water. Stream 15b products include but are not limited to soy whey protein, BBI, KTI and, other proteins.

Step 16 (See FIG. 4B) a heat treatment and flash cooling step can start with soy whey protein, BBI, KTI and, other proteins from stream 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

Embodiment 18 starts with Step 0 (See FIG. 4A) the whey protein pretreatment can start with feed streams including but not limited to isolated soy protein (ISP) molasses, ISP whey, soy protein concentrate (SPC) molasses, SPC whey, functional soy protein concentrate (FSPC) whey, and combinations thereof. Processing aids that can be used in the whey protein pretreatment step include but are not limited to, acids, bases, sodium hydroxide, calcium hydroxide, hydrochloric acid, water, steam, and combinations thereof. The pH of step 0 after the pH is adjusted can be between about 3.0 and about 6.0, or between about 3.5 and about 5.5, or about 5.3. The temperature can be between about 70° C. and about 95° C., or about 85° C. Temperature hold times can vary between about 0 minutes to about 20 minutes, or about 10 minutes. After the hold time, the stream is passed through a centrifugal separation step, typically an intermittent discharge disc clarifying centrifuge, in order to separate the precipitate from the whey stream. Products from the whey protein pretreatment include but are not limited to soluble components in the aqueous phase of the whey stream (pre-treated soy whey) (molecular weight of equal to or less than about 50 kiloDalton (kDa)) in stream 0a and insoluble large molecular weight proteins (between about 300 kDa and between about 50 kDa) in stream 0b, such as pre-treated soy whey, storage proteins, and combinations thereof.

Step 1 (See FIG. 4A) Microbiology reduction can start with the product of the whey protein pretreatment step, including but not limited to pre-treated soy whey. This step involves microfiltration of the pre-treated soy whey. Process variables and alternatives in this step include but are not limited to, centrifugation, dead-end filtration, heat sterilization, ultraviolet sterilization, microfiltration, crossflow membrane filtration, and combinations thereof. Crossflow membrane filtration includes but is not limited to spiral-wound, plate and frame, hollow fiber, ceramic, dynamic or rotating disk, nanofiber, and combinations thereof. The pH of step 1 can be between about 2.0 and about 12.0, or between about 3.5 and about 5.5, or about 5.3. The temperature can be between about 5° C. and about 90° C., or between about 25° C. and about 75° C., or about 50° C. Products from step 1 include but are not limited to storage proteins, microorganisms, silicon, and combinations thereof in stream 1a and purified pre-treated soy whey in stream 1b.

Step 2 (See FIG. 4A) a water and mineral removal can start with the purified pre-treated soy whey from stream 1b. It includes a nanofiltration step for water removal and partial mineral removal. Process variables and alternatives in this step include but are not limited to, crossflow membrane filtration, reverse osmosis, evaporation, nanofiltration, and combinations thereof. Crossflow membrane filtration includes but is not limited to: spiral-wound, plate and frame, hollow fiber, ceramic, dynamic or rotating disk, nanofiber, and combinations thereof. The pH of step 2 can be between about 2.0 and about 12.0, or between about 3.5 and about 5.5, or about 5.3. The temperature can be between about 5° C. and about 90° C., or between about 25° C. and about 75° C. or about 50° C. Products from this water removal step include but are not limited to purified pre-treated soy whey in stream 2a and water, some minerals, monovalent cations and combinations thereof in stream 2b.

Step 3 (See FIG. 4A) the mineral precipitation step can start with purified pre-treated soy whey from stream 2a. It includes a precipitation step by pH and/or temperature change. Process variables and alternatives in this step include but are not limited to, an agitated or recirculating reaction tank. Processing aids that can be used in the mineral precipitation step include but are not limited to, acids, bases, calcium hydroxide, sodium hydroxide, hydrochloric acid, sodium chloride, phytase, and combinations thereof. The pH of step 3 can be between about 2.0 and about 12.0, or between about 6.0 and about 9.0, or about 8.0. The temperature can be between about 5° C. and about 90° C., or between about 25° C. and about 75° C., or about 50° C. The pH hold times can vary between about 0 minutes to about 60 minutes, or between about 5 minutes and about 20 minutes, or about 10 minutes. The product of stream 3 is a suspension of purified pre-treated soy whey and precipitated minerals.

Step 4 (See FIG. 4A)—the mineral removal step can start with the suspension of purified pre-treated whey and precipitated minerals from stream 3. It includes a centrifugation step. Process variables and alternatives in this step include but are not limited to, centrifugation, filtration, dead-end filtration, crossflow membrane filtration and combinations thereof. Crossflow membrane filtration includes but is not limited to: spiral-wound, plate and frame, hollow fiber, ceramic, dynamic or rotating disk, nanofiber, and combinations thereof. Products from the mineral removal step include but are not limited to a de-mineralized pre-treated whey in stream 4a and insoluble minerals with some protein mineral complexes in stream 4b.

Step 5 (See FIG. 4B) the protein separation and concentration step can start with purified pre-treated whey from stream 4a. It includes an ultrafiltration step. Processing aids that can be used in the ultrafiltration step include but are not limited to, acids, bases, calcium hydroxide, sodium hydroxide, hydrochloric acid, and combinations thereof. Process variables and alternatives in this step include but are not limited to, crossflow membrane filtration, ultrafiltration, and combinations thereof. Crossflow membrane filtration includes but is not limited to: spiral-wound, plate and frame, hollow fiber, ceramic, dynamic or rotating disk, nanofiber, and combinations thereof. The pH of step 5 can be between about 2.0 and about 12.0, or between about 6.0 and about 9.0, or about 8.0. The temperature can be between about 5° C. and about 90° C., or between about 25° C. and about 75° C., or about 50° C. Products from stream 5a include but are not limited to, soy whey protein, BBI, KTI, storage proteins, other proteins and combinations thereof. Products from stream 5b include but are not limited to, peptides, soy oligosaccharides, minerals and combinations thereof.

Step 6 (See FIG. 4B) the protein washing and purification step can start with soy whey protein, BBI, KTI, storage proteins, other proteins or purified pre-treated whey from stream 5a. It includes a diafiltration step. Process variables and alternatives in this step include but are not limited to, reslurrying, crossflow membrane filtration, ultrafiltration, water diafiltration, buffer diafiltration, and combinations thereof. Crossflow membrane filtration includes but is not limited to: spiral-wound, plate and frame, hollow fiber, ceramic, dynamic or rotating disk, nanofiber, and combinations thereof. Processing aids that can be used in the protein washing and purification step include but are not limited to, water, steam, and combinations thereof. The pH of step 6 can be between about 2.0 and about 12.0, or between about 6.0 and about 9.0, or about 7.0. The temperature can be between about 5° C. and about 90° C., or between about 25° C. and about 75° C., or about 50° C. Products from stream 6a include but are not limited to, soy whey protein, BBI, KTI, storage proteins, other proteins, and combinations thereof. Products from stream 6b include but are not limited to, peptides, soy oligosaccharides, water, minerals, and combinations thereof.

Step 15 (See FIG. 48) a water removal step can start with soy whey protein, BBI, KTI and, other proteins from stream 6a. It includes an evaporation step. Process variables and alternatives in this step include but are not limited to, evaporation, nanofiltration, reverse osmosis, and combinations thereof. Products from stream 15a include but are not limited to, water. Stream 15b products include but are not limited to soy whey protein, BBI, KTI and, other proteins.

Step 16 (See FIG. 4B) a heat treatment and flash cooling step can start with soy whey protein, BBI, KTI and, other proteins from stream 15b. It includes an ultra high temperature step. Process variables and alternatives in this step include but are not limited to, heat sterilization, evaporation, and combinations thereof. Processing aids that can be used in this heat treatment and flash cooling step include but are not limited to, water, steam, and combinations thereof. The temperature of the heating step can be between about 129° C. and about 160° C., or about 152° C. Temperature hold time can be between about 8 seconds and about 15 seconds, or about 9 seconds. Upon flash cooling, the temperature can be between about 50° C. and about 95° C., or about 82° C. Products from stream 16 include but are not limited to, soy whey protein.

Finally, Step 17 (See FIG. 4B) a drying step can start with soy whey protein, BBI, KTI and, other proteins from stream 16. It includes a drying step. The liquid feed temperature can be between about 50° C. and about 95° C., or about 82° C. The inlet temperature can be between about 175° C. and about 370° C., or about 290° C. The exhaust temperature can be between about 65° C. and about 98° C., or about 88° C. Products from stream 17a include but are not limited to, water. Products from stream 17b include but are not limited to, soy whey protein which includes, BBI, KTI and, other proteins.

### Embodiments Directed to Recovery of Sugars:

Embodiment 19 encompasses Step 7 (See FIG. 4C) a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., or between about 25° C. and about 75° C., or about 50° C. Products from stream 7a include but are not limited to, peptides, soy oligosaccharides, water, minerals, and combinations thereof. Products from stream 7b include but are not limited to, water, minerals, and combinations thereof.

Embodiment 20 starts with Step 7 (See FIG. 4C) a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., or between about 25° C. and about 75° C., or about 50° C. Products from stream 7a include but are not limited to, peptides, soy oligosaccharides, water, minerals, and combinations thereof. Products from stream 7b include but are not limited to, water, minerals, and combinations thereof.

Finally, Step 11 (See FIG. 4C) a water removal step can start with soy oligosaccharides such as, raffinose, stachyose, verbascose, and combinations thereof from stream 7a. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., or about 60° C. Products from stream 11a include but are not limited to, water. Products from stream 11b include but are not limited to, soy oligosaccharides.

Embodiment 21 starts with Step 7 (See FIG. 4C) a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., or between about 25° C. and about 75° C., or about 50° C. Products from stream 7a include but are not limited to, peptides, soy oligosaccharides, water, minerals, and combinations thereof. Products from stream 7b include but are not limited to, water, minerals, and combinations thereof.

Finally, Step 8 (See FIG. 4C) a mineral removal step can start with peptides, soy oligosaccharides, water, minerals, and combinations thereof from stream 7a. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, or between about 6.0 and about 9.0, or about 7.0. The temperature can be between about 5° C. and about 90° C., or between about 25° C. and about 50° C., or about 40° C. Products from stream 8a include but are not limited to, de-mineralized soy oligosaccharides with conductivity between about 10 milli Siemens/centimeter (mS/cm) and about 0.5 mS/cm, or about 2 mS/cm. Products from stream 8b include but are not limited to, minerals, water, and combinations thereof.

Embodiment 22 starts with Step 7 (See FIG. 4C) a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., or between about 25° C. and about 75° C., or about 50° C. Products from stream 7a include but are not limited to, peptides, soy oligosaccharides, water, minerals, and combinations thereof. Products from stream 7b include but are not limited to, water, minerals, and combinations thereof.

Step 8 (See FIG. 4C) a mineral removal step can start with peptides, soy oligosaccharides, water, minerals, and combinations thereof from stream 7a. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, or between about 6.0 and about 9.0, or about 7.0. The temperature can be between about 5° C. and about 90° C., or between about 25° C. and about 50° C., or about 40° C. Products from stream 8a include but are not limited to, de-mineralized soy oligosaccharides with conductivity between about 10 milli Siemens/centimeter (mS/cm) and about 0.5 mS/cm, or about 2 mS/cm. Products from stream 8b include but are not limited to, minerals, water, and combinations thereof.

Finally, Step 11 (See FIG. 4C) a water removal step can start with soy oligosaccharides such as, raffinose, stachyose, verbascose, and combinations thereof from stream 8a. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., or about 60° C. Products from stream 11a include but are not limited to, water. Products from stream 11b include but are not limited to, soy oligosaccharides.

Embodiment 23 starts with Step 7 (See FIG. 4C) a water removal step can start with peptides, soy oligosaccharides, water, minerals, and combinations thereof from stream 5b and/or stream 6b. Soy oligosaccharides include but are not limited to sucrose, raffinose, stachyose, verbascose, monosaccharides, and combinations thereof. It includes a nanofiltration step. Process variables and alternatives in this step include but are not limited to, reverse osmosis, evaporation, nanofiltration, water diafiltration, buffer diafiltration, and combinations thereof. The pH of step 7 can be between about 2.0 and about 12.0, or between about 6.0 and about 9.0, or about 7.0. The temperature can be between about 5° C. and about 90° C., or between about 25° C. and about 75° C., or about 50° C. Products from stream 7a include but are not limited to, peptides, soy oligosaccharides, water, minerals, and combinations thereof. Products from stream 7b include but are not limited to, water, minerals, and combinations thereof.

Step 8 (See FIG. 4C) a mineral removal step can start with peptides, soy oligosaccharides, water, minerals, and combinations thereof from stream 7a. It includes an electrodialysis membrane step. Process variables and alternatives in this step include but are not limited to, ion exchange columns, chromatography, and combinations thereof. Processing aids that can be used in this mineral removal step include but are not limited to, water, enzymes, and combinations thereof. Enzymes include but are not limited to protease, phytase, and combinations thereof. The pH of step 8 can be between about 2.0 and about 12.0, or between about 6.0 and about 9.0, or about 7.0. The temperature can be between about 5° C. and about 90° C., or between about 25° C. and about 50° C., or about 40° C. Products from stream 8a include but are not limited to, de-mineralized soy oligosaccharides with conductivity between about 10 milli Siemens/centimeter (mS/cm) and about 0.5 mS/cm, or about 2 mS/cm. Products from stream 8b include but are not limited to, minerals, water, and combinations thereof.

Step 9 (See FIG. 4C) a color removal step can start with de-mineralized soy oligosaccharides from stream 8a. It utilizes an active carbon bed. Process variables and alternatives in this step include but are not limited to, ion exchange. Processing aids that can be used in this color removal step include but are not limited to, active carbon, ion exchange resins, and combinations thereof. The temperature can be between about 5° C. and about 90° C., or about 40° C. Products from stream 9a include but are not limited to, color compounds. Stream 9b is a decolored solution. Products from stream 9b include but are not limited to, soy oligosaccharides, and combinations thereof.

Step 10 (See FIG. 4C) a soy oligosaccharide fractionation step can start with soy oligosaccharides, and combinations thereof from stream 9b. It includes a chromatography step. Process variables and alternatives in this step include but are not limited to, chromatography, nanofiltration, and combinations thereof. Processing aids that can be used in this soy oligosaccharide fractionation step include but are not limited to acid or base to adjust the pH as one skilled in the art would know, based on the resin used. Products from stream 10a include but are not limited to, soy oligosaccharides. Products from stream 10b include but are not limited to soy oligosaccharides.

Finally, Step 11 (See FIG. 4C) a water removal step can start with soy oligosaccharides such as, raffinose, stachyose, verbascose, and combinations thereof from stream 10a. It includes an evaporation step. Process variables and alternatives in this step include but are not limited to, evaporation, reverse osmosis, nanofiltration, and combinations thereof. Processing aids that can be used in this water removal step include but are not limited to, defoamer, steam, vacuum, and combinations thereof. The temperature can be between about 5° C. and about 90° C., or about 60° C. Products from stream 11a include but are not limited to, water. Products from stream 11b include but are not limited to, soy oligosaccharides.

### DEFINITIONS

To facilitate understanding of the invention, several terms are defined below.

The term “acid soluble” as used herein refers to a substance having a solubility of at least about 80% with a concentration of 10 grams per liter (g/L) in an aqueous medium having a pH of from about 2 to about 7.

The terms “soy protein isolate” (SPI) or “isolated soy protein,” (ISP) as used herein, refer to a soy material having a protein content of at least about 90% soy protein on a moisture free basis.

The term “other proteins” as used herein referred to throughout the application are defined as including but not limited to: lunasin, lectins, dehydrins, lipoxygenase, and combinations thereof.

The term “soy oligosaccharide” is defined as including but not limited to sugar. Sugar is defined as including but not limited to sucrose, raffinose, stachyose, verbascose, monosaccharides, and combinations thereof.

The term “soy whey protein” as used herein is defined as including protein soluble at those pHs where soy storage proteins are typically insoluble, including but not limited to BBI, KTI, lunasin, lipoxygenase, dehydrins, lectins, peptides, and combinations thereof. Soy whey protein may further include storage proteins.

The term “subject” or “subjects” as used herein refers to a mammal (preferably a human), bird, fish, reptile, or amphibian, in need of treatment for a pathological state, which pathological state includes, but is not limited to, diseases associated with muscle, uncontrolled cell growth, autoimmune diseases, and cancer.

The term “processing stream” as used herein refers to the secondary or incidental product derived from the process of refining a whole legume or oilseed, including an aqueous or solvent stream, which includes, for example, an aqueous soy extract stream, an aqueous soymilk extract stream, an aqueous soy whey stream, an aqueous soy molasses stream, an aqueous soy protein concentrate soy molasses stream, an aqueous soy permeate stream, and an aqueous tofu whey stream, and additionally includes soy whey protein, for example, in both liquid and dry powder form, that can be recovered as an intermediate product in accordance with the methods disclosed herein.

When introducing elements of the present invention or the preferred embodiments(s) thereof, the articles “a,” “an,” “the” and “said” are intended to mean that there are one or more of the elements. The terms “comprising,” “including” and “having” are intended to be inclusive and mean that there may be additional elements other than the listed elements.

As various changes could be made in the above compounds, products and methods without departing from the scope of the invention, it is intended that all matter contained in the above description and in the examples given below, shall be interpreted as illustrative and not in a limiting sense.

### EXAMPLES

### Example 1

### Recovery and Fractionation of Soy Whey Protein from Aqueous Soy Whey Using Novel Membrane Process

145 liters of aqueous raw soy whey (not pre-treated) with a total solids content of 3.7% and dry basis protein content of 19.8% was microfiltered using two different membranes in an OPTISEP® 7000 module, manufactured by SmartFlow Technologies. The first membrane, BTS-25, was a polysulfone construction with 0.5 um pore size manufactured by Pall. Aqueous soy whey was concentrated to a 1.6× factor, at an average flux of 30 liters/meter²/hr (LMH). The concentrated aqueous soy whey was then passed through a modified polysulfone microfiltration membrane, MPS 0.45, manufactured by Pall. The aqueous soy whey was concentrated from 1.6× to 11× at an average flux of 28 LMH.

Permeate from the microfiltration process, 132 liters total, was then introduced into an OPTISEP® 7000 module with ultrafiltration membranes, RC100, which are 100 kDa regenerated cellulose membranes manufactured by Microdyn-Nadir. The microfiltered aqueous soy whey was concentrated to about 20× using a 20 L tank setup at an average flux of 30 LMH before being transferred to a 5 L tank setup in order to minimize the hold-up volume of the system. In the smaller tank, the aqueous soy whey was concentrated from 20× to 66× at an average flux rate of 9 LMH, reaching a final retentate volume of 2 liters. The final retentate was 24.0% total solids, and 83.0% dry basis protein content.

128 liters of sugar and mineral enriched RC100 permeate was then introduced into an OPTISEP® 7000 module with polysulfone thin film nanofiltration membranes with a 35% NaCl rejection rate, NF20, manufactured by Sepro. The feed was concentrated 18× at an average flux rate of 4.7 LMH. The retentate from this process step, 9 liters, was enriched in the various sugar species. The permeate stream from the NF20 separation process, 121 liters, contained the minerals and water.

The permeate of the NF20 process was then introduced into an OPTISEP® 3000 module with thin film reverse osmosis membranes with a 98.2% NaCl rejection rate, SG, manufactured by GE. The feed was concentrated 12× at an average flux rate of 8 LMH. The permeate of the SG membrane, 9.2 liters, consisted primarily of water, suitable for re-use in a process with minimal further treatment. The retentate of the SG process, 0.8 liters, consisted predominantly of a concentrated mineral fraction.

### Example 2

### Recovery and Fractionation of Soy Whey Protein from Soy Molasses Using Novel Membrane Process

61.7 liters of soy molasses with a total solids content of 62.7% and dry basis protein content of 18.5% was diluted with 61.7 liters of water prior to microfiltration. The diluted soy molasses was then microfiltered using an OPTISEP® 7000 module, manufactured by SmartFlow Technologies. The diluted soy molasses passed through a modified polysulfone microfiltration membrane, MPS 0.45, manufactured by Pall. The diluted soy molasses was concentrated to a 1.3× factor, at an average flux of 6 liters/meter²/hr (LMH).

Permeate from the microfiltration process, 25 liters total, was then introduced into an OPTISEP® 7000 module with ultrafiltration membranes, RC100, which are 100 kDa regenerated cellulose membranes manufactured by Microdyn-Nadir. The microfiltered diluted soy molasses was diafiltered with 2 volumes of water prior to being concentrated to 7.6× at an average flux of 20 LMH, reaching a final retentate volume of 2 liters. The final retentate was 17.5% total solids, and 22.0% dry basis protein content.

72 liters of sugar and mineral enriched RC100 permeate was then introduced into an OPTISEP® 7000 module with polysulfone thin film nanofiltration membranes with a 35% NaCl rejection rate, NF20, manufactured by Sepro. The feed was concentrated 3× at an average flux rate of 4.0 LMH. The retentate from this process step, 23 liters, was enriched in the various sugar species. The permeate stream from the NF20 separation process, 48 liters, contained the minerals and water.

A portion of the permeate of the NF20 process, 10 liters, was then introduced into an OPTISEP® 3000 module with thin film reverse osmosis membranes with a 98.2% NaCl rejection rate, SG, manufactured by GE. The feed was concentrated 6.7× at an average flux rate of 7.9 LMH. The permeate of the SG membrane, 8.5 liters, consisted primarily of water, suitable for re-use in a process with minimal further treatment. The retentate of the SG process, 1.5 liters, consisted predominantly of a concentrated mineral fraction.

### Example 3

### Capture of Bulk Soy Whey Protein from Defatted Soy Flour Extract

Defatted soy flour (DSF) was extracted by adding a 15:1 ratio of water to DSF at a pH of 7.8 and stirring for 20 minutes prior to filtration. The extract was microfiltered using an OPTISEP® 800 module, manufactured by SmartFlow Technologies. The microfiltration membrane, MMM-0.8, was a polysulfone and polyvinylpropylene construction with 0.8 um pore size manufactured by Pall. Aqueous soy extract was concentrated to a 2.0× factor, at an average flux of 29 liters/meter²/hr (LMH). Permeate from the microfiltration process was then introduced into an OPTISEP® 800 module with ultrafiltration membranes, RC100, which are 100 kDa regenerated cellulose membranes manufactured by Microdyn-Nadir. The microfiltered aqueous soy extract was concentrated to about 6.3× at an average flux rate of 50 LMH. The final retentate measured 84.7% dry basis protein content.

### Example 4

### Capture of Bulk Soy Whey Protein Using Ion Exchange Chromatography (to Establish Conditions for Use in Continuous Separation Technology CSEP (Simulated Moving Bed Chromatography))

CSEP experiments were performed by passing feed material (soy whey) through a column (ID 1.55 cm, length 9.5 cm, volume 18 mL) packed with SP GibcoCel resin. The column was connected to a positive displacement pump and samples of flow through and eluates were collected at the outlet of the column. Different experimental conditions were used to determine the effect of feed concentration, feed flow rate and elution flow rate on the binding capacity of the resin.

Feed Concentration

Soy whey was prepared from the defatted soy flake. Briefly, one part of defatted flake was mixed with 15 parts of water at 32° C. The pH of the solution was adjusted to 7.0 using 2 M NaOH and proteins were extracted into the aqueous phase by stirring the solution for 15 min. The protein extract was separated from the insoluble material by centrifugation at 3000×g for 10 min. The pH of the collected supernatant was adjusted to 4.5 using 1 M HCl and the solution was stirred for 15 min followed by heating to a temperature of 57° C. This treatment resulted in precipitation of the storage proteins while the whey proteins remained soluble. The precipitated proteins were separated from the whey by centrifugation at 3000×g for 10 min.

In some cases, the soy whey was concentrated using a Lab-Scale Amicon DC-10LA ultrafiltration unit and Amicon 3K membrane. Prior to ultrafiltration, pH of soy whey was adjusted to 5.5 with 2 M NaOH to avoid membrane fouling at acidic conditions. 10 L of whey was processed with the flux at ˜100 mL/min. Once the concentration factor of 5 in the retentate was reached, both retentate and permeate streams were collected. Soy whey concentrates 2.5×, 3×, and 4× were prepared by mixing a known amount of permeate and 5× whey concentrate. The pH of all soy concentrates was readjusted if necessary to 4.5.

Feed Flow Rate

During dynamic adsorption, as fluid flows through the resin bed, the proteins are adsorbed by the resin and reach equilibrium with the liquid phase. As the whey is loaded onto the column, the bound protein band extends down the column and reaches equilibrium with the liquid phase. When the resin is saturated with adsorbed proteins, the concentration of the proteins in the liquid phase exiting the column will be similar to the protein concentration in the feed. The curve describing the change in the flow through concentration compared to the feed concentration with the passage of fluid is the breakthrough curve. The concentration of protein in the solid phase increases as the breakthrough curve is developed, and the adsorption wave moves through the bed. As more fluid is passed through the bed, the flow through concentration increases asymptotically to the incoming fluid stream and at the same time a similar phenomena is achieved with the solid phase.

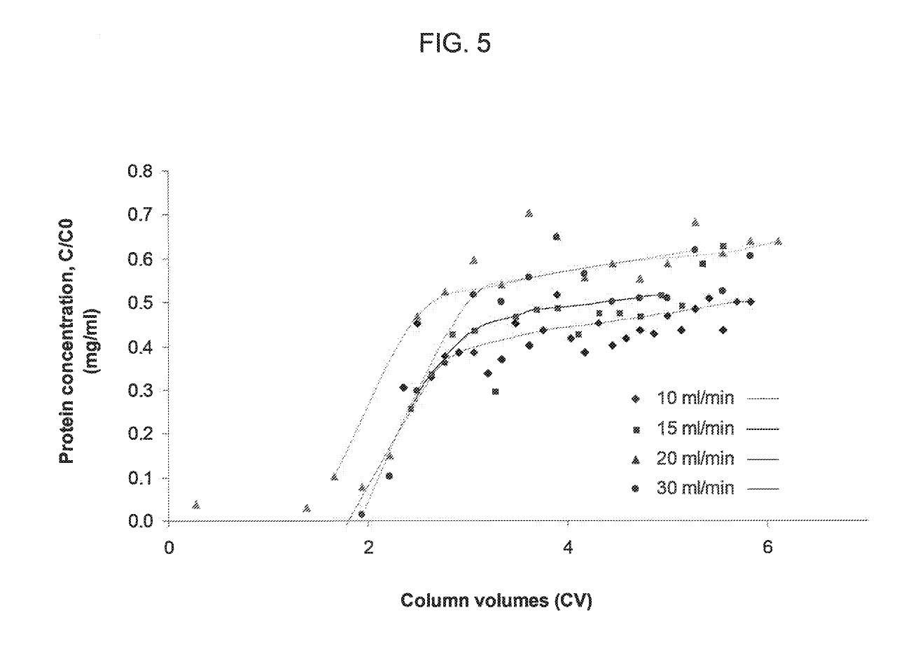

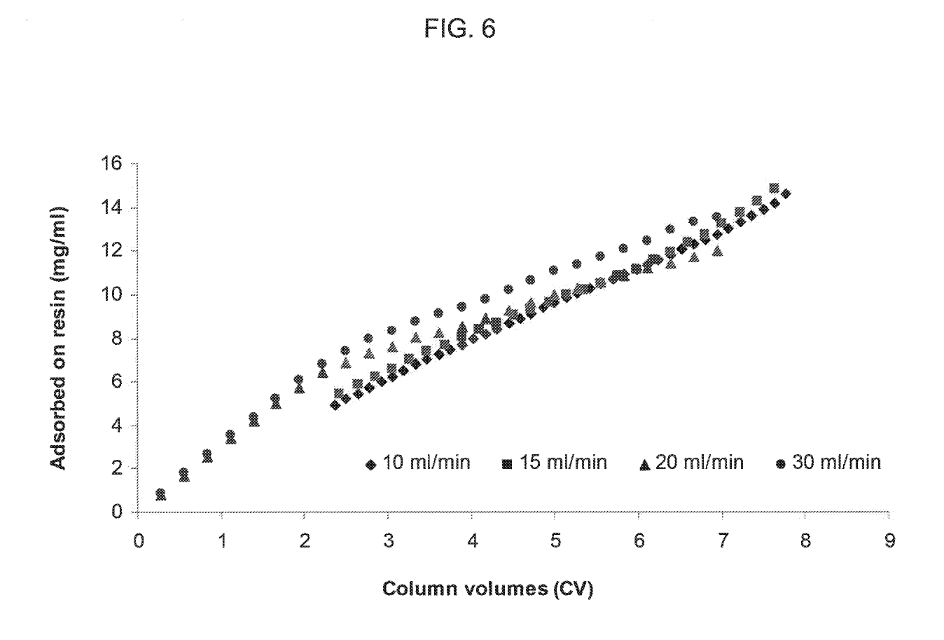

The flow through protein concentration data at three different linear velocity rates were plotted against the column volumes of soy whey loaded (see FIG. 5). These data indicated that increasing the linear flow rate of loading by a factor of 3 resulted in about 10% increase in the unabsorbed proteins in the flow through after loading 6 column volumes of soy whey. Therefore the linear flow rate does not significantly impact the adsorption characteristics of the soy whey proteins with the SP Gibco resin. The equilibrium adsorption data (see FIG. 6) showed that the soy whey protein adsorbed on the resin (calculated using mass balance of protein feed to the system and the protein concentration in the flow through, in equilibrium with the protein in the liquid stream, and plotted against the column volumes passed through the resin bed) varied little with flow rate of the feed at the fluxes tested.

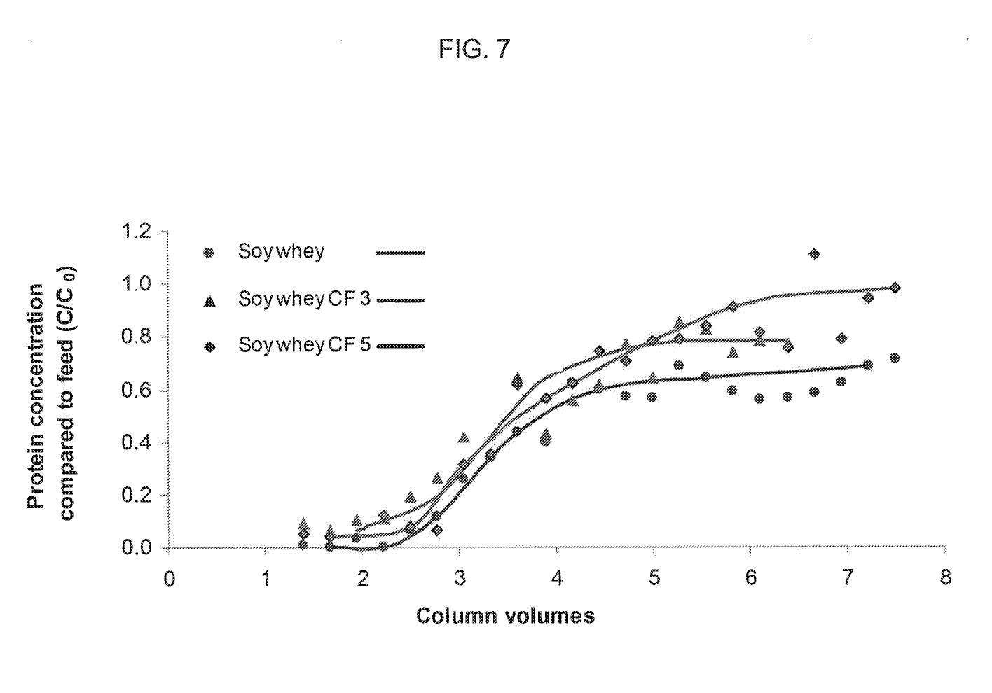

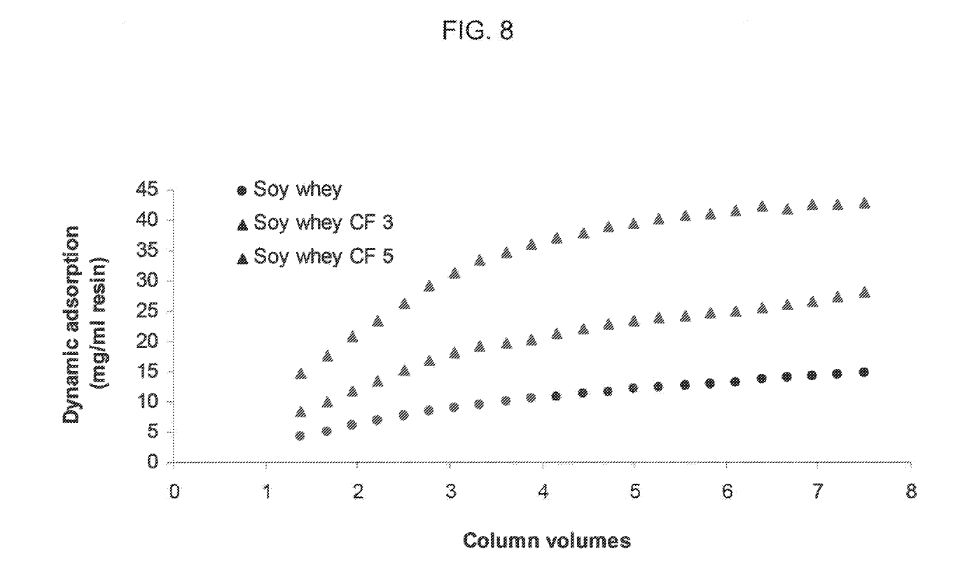

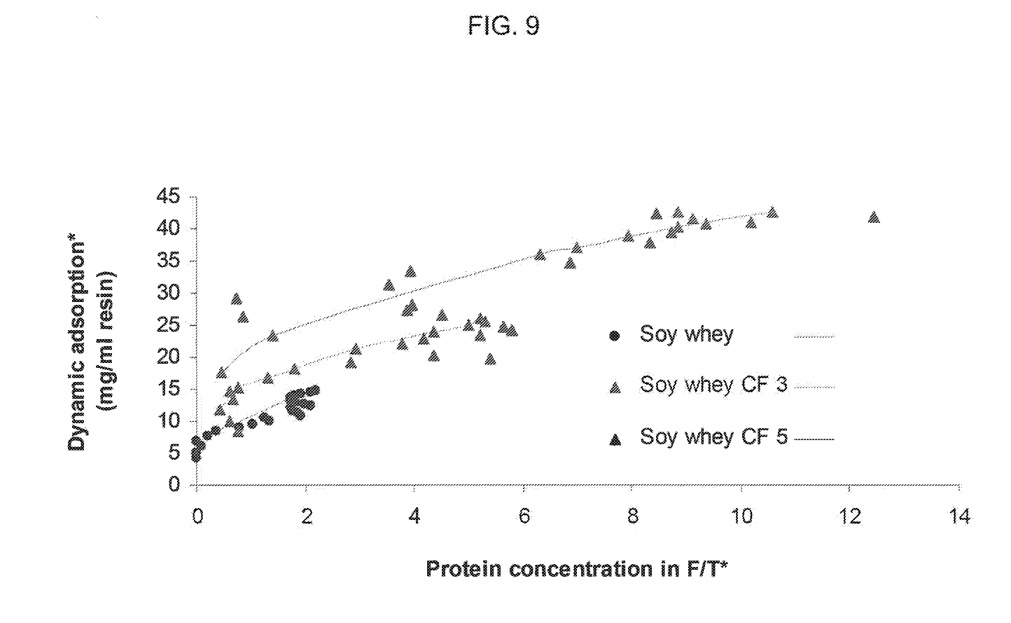

The profile of the breakthrough curve, where soy whey and soy whey concentrated by a factor of 3 and 5 was applied to an SP Gibco resin bed at 15 mL/min (8.5 cm/min linear flow rate), was similar with all three concentrations (see FIG. 7). This result indicated that as the feed protein concentration was increased the resin reached equilibrium with the protein concentration in the liquid stream by striving to reach maximum capacity. This increased adsorption is depicted in FIG. 8 where the protein concentration in the solid phase in equilibrium with the liquid phase has been plotted against the column volumes of soy whey passed through the bed. These data show that the protein adsorbed by the resin significantly increased with soy whey concentration factor, and hence the protein concentration in the soy whey (see FIG. 8). FIG. 9 shows the equilibrium characteristics of the resin and the flow through. This chart shows that as the number of column volumes were passed through the bed, the adsorption of proteins in the resin phase increased asymptotically but the protein content in the flow through also increased. Adsorption capacity can be increased by using concentrated whey and loading at high column volumes but this resulted in a relatively high protein content in the flow through. However, the high protein content in the flow through was minimized by counter current operation using a 2-stage adsorption strategy.

Based on the dynamic adsorption data (see FIG. 9), loading whey concentrated by factor >5 to achieve a protein concentration of >11 mg/mL and loading about 3.5 column volumes resulted in about 35 mg protein adsorbed per mL of resin, and the equilibrium protein concentration in the flow through was about 6.8 mg/mL. Presenting this primary flow through to another resin bed in a second pass (loading about 3.5 column volumes) resulted in a protein concentration in the flow through of about 1.3 mg/mL. Therefore, using two passes of adsorption and operating the chromatography in counter current mode resulted in adsorption of about 90% of the available soy protein that could be absorbed from soy whey at pH 4.5.

Elution Flow Rate

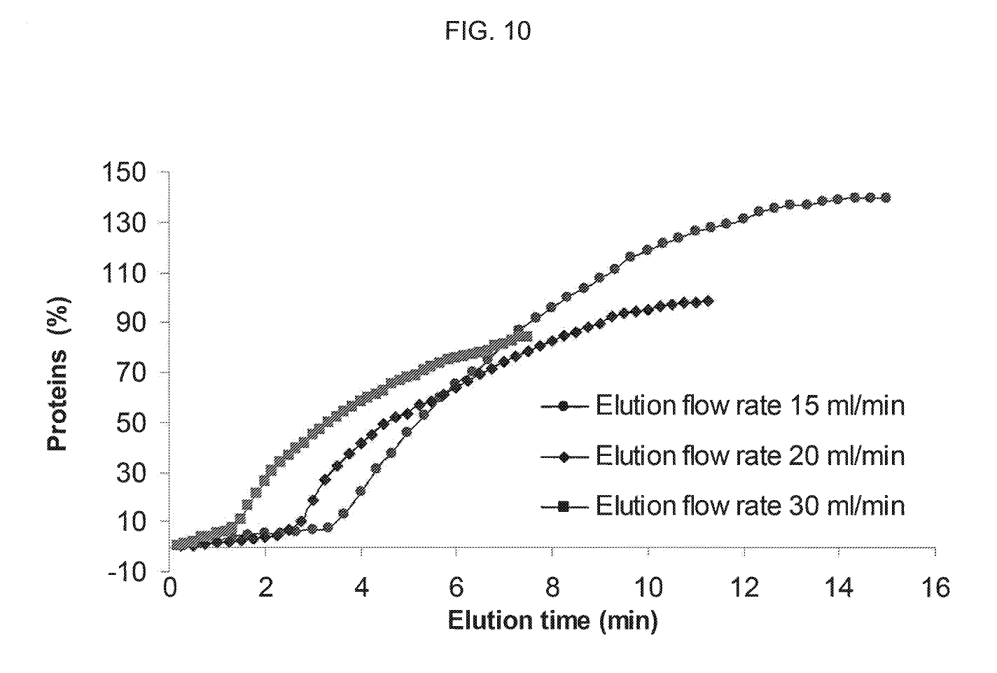

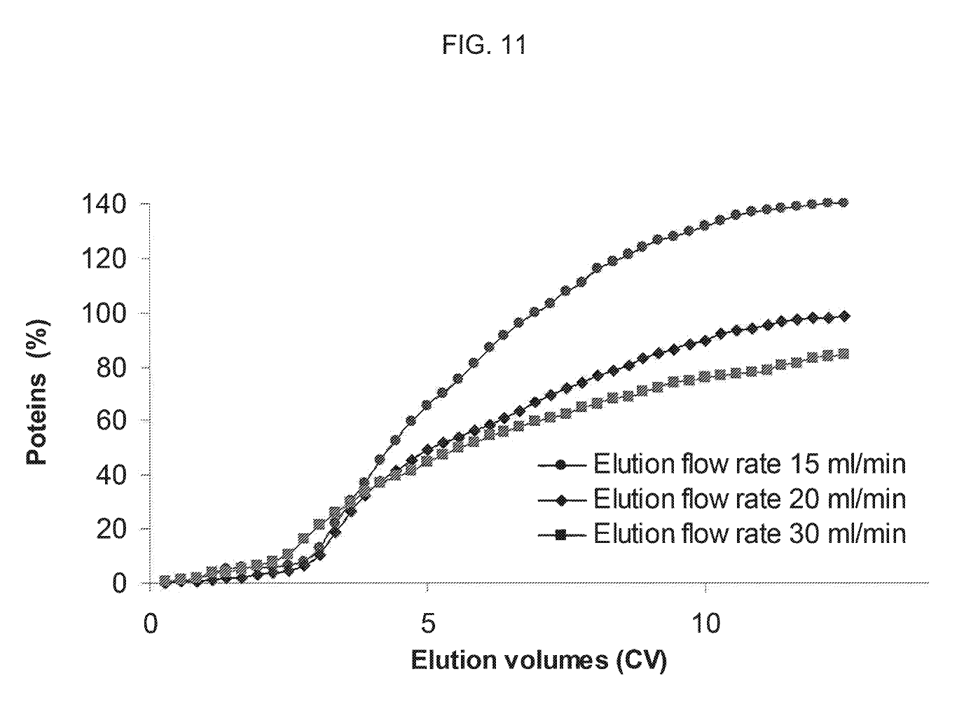

The effect of elution flow rate was investigated at three different flow rates and the recovery data are shown in Table 3. The recovery of protein at low flow rates in duplicate experiments resulted in recoveries of over 164% and 200%. The data indicate that eluting at 20 and 30 mL/min (11.3 and 17.0 cm/min, respectively) did not significantly affect the recoveries. Moreover, operating at higher flow rates achieved much faster elution (see FIG. 10), however at these higher flow rates a larger column volume of eluate was required to complete the elution (see FIG. 11). The need for a larger column volume of eluate was overcome by recycling the eluate which also reduced the total volume required for elution and also presented a more concentrated protein stream to the downstream ultrafiltration unit, reducing the membrane area needed for protein concentration.

TABLE 3
Elution and recovery of bound soy whey proteins at three different
flow rates.

| | ELUTION FLOW RATES | | |
|---|---|---|---|
| | 15 mL/min | 20 mL/min | 30 mL/min |
| Protein adsorbed | 75.4 ± 4.4 | 70.8 ± 2.7 | 72.9 ± 4.8 |
| (mg) | | | |
| Protein eluted | 139.7 ± 22.9 | 73.2 ± 1.5 | 68.4 ± 6.8 |
| (mg) | | | |
| Recovery (%) | 184.2 ± 19.7 | 103.4 ± 6.1 | 93.8 ± 15.6 |

Protein adsorption was calculated as the difference in the protein content in the feed and flow through by mass balance.

### Example 5

### Capture of Bulk Soy Whey Protein from a Pre-Treated Whey Process (PT)

The feed stream to the process, pre-treated whey protein, (also referred to PT whey) had approximately 1.4%-2.0% solids. It was comprised of approximately 18% minerals, 18% protein, and 74% sugars and other materials. Implementation of a Nanofiltration (NF) process allowed for water removal while retaining most of the sugars and protein, and other solid material, in the process to be recovered downstream. The NF membranes (Alfa Laval NF99 8038/48) for the trial were polyamide type thin film composite on polyester membranes with a 2 kDa molecular weight cutoff (MWCO) that allowed water, monovalent cations, and a very small amount of sugars and protein to pass through the pores. The membrane housing held 3 membrane elements. Each element was 8 inches in diameter and had 26.4 square meters of membrane surface area. The total membrane surface area for the process was 79.2 square meters. These membranes were stable up to 1 bar of pressure drop across each membrane element. For the entire module containing 3 membrane elements, a pressure drop of 3 bar was the maximum allowable. The NF feed rate of PT whey was approximately 2,500 L/hour. The temperature of this feed was approximately 45-50° C., and the temperature of the NF operation was regulated to be in this range using cooling water. Initial product flux rates were approximately 16-22 liters per meter squared per hour (LMH). The feed pressure at the inlet of the module was approximately 6 bar. Through the duration of the 6 hour run, the flux dropped as a result of fouling. The feed pressure was increased incrementally to maintain higher flux, but as fouling occurred, the pressure was increased to the maximum, and the flux slowly tapered from that point. Volumetric concentration factors were between 2× and approximately 4×.

A Precipitation step was performed to separate, e.g., phosphorous and calcium salts and complexes from the PT whey. Precipitation conditions were at pH 9 while maintaining the temperature at 45° C. with a residence time of approximately 15 minutes. The precipitation process occurred in a 1000 liter. This tank had multiple inlets and outlets where materials can be piped into and out of it. A small centrifugal pump circulated product out of the tank and back into the side of the tank to promote agitation and effective mixing of the 35% NaOH added to the system to maintain the target pH. This pump also sent product into the centrifuge when one of the T-valves connected to this recirculation loop was opened. Concentrated PT whey from the NF was fed directly into the top of the tank. 35% NaOH was connected into the feed line from the NF in order to control the pH at the target value. PT whey was fed into this mixing tank at approximately 2,500 L/hour and fed out at the same rate.

In following process step, an Alfa Laval Disc Centrifuge (Clara 80) with intermittent solids ejection system was used to separate precipitated solids (including insoluble soy fiber, insoluble soy protein) from the rest of the sugar- and protein-containing whey stream. In this process, concentrated PT whey from the precipitation tank was pumped into a disc-centrifuge where this suspension was rotated and accelerated by centrifugal force. The heavier fraction (precipitated solids) settles on the walls of the rotating centrifuge bowl with the lighter fraction (soluble liquid) was clarified through the use of disc-stacks and continuously discharged for the next step of the process. The separated precipitated solids was discharged at a regular interval (typically between 1 and 10 minutes). The clarified whey stream was less then 0.2% solids on a volumetric basis. The continuous feed flow rate was approximately 2.5 m3/hr, with a pH of 9.0 and 45° C. The insoluble fraction reached Ash=30-60%; Na=0.5-1.5% dry basis, K=1.5-3% dry basis, Ca=6-9% dry basis, Mg=3-6% dry basis, P=10-15% dry basis, Cl=1-2% dry basis, Fe, Mn, Zn, Cu <0.15% dry basis. Changes to the soluble fraction were as follows: Phytic acid was approximately 0.3% dry basis (85% reduction, P=0.2-0.3% dry basis (85-90% reduction), Ca=0.35-0.45% dry basis (80-85% reduction), Mg=0.75-0.85% dry basis (15-20% reduction).

The next step was an Ultrafiltration (UF) membrane. Protein was concentrated by being retained by a membrane while other smaller solutes pass into the permeated stream. From the centrifuge a diluted stream the containing protein, minerals and sugars was fed to the UF. The UF equipment and the membrane were supplied from Alfa Laval while the CIP chemicals came from Ecolab, Inc. The tested membrane, GR70PP/80 from Alfa-Laval, had a MWCO of 10 kDa and was constructed of polyethersulfone (PES) cast onto a polypropylene polymer backing. The feed pressure varied throughout the trial from 1-7 bar, depending upon the degree of fouling of the membranes. The temperature was controlled to approximately 65° C. The system was a feed and bleed setup, where the retentate was recycled back to the feed tank while the permeate proceeded on to the next step in the process. The system was operated until a volume concentration factor of 30× was reached. The feed rate to the UF was approximately 1,600 L/hour. The setup had the ability to house 3 tubes worth of 6.3″ membrane elements. However, only one of the three tubes was used. The membrane skid had an automatic control system that allowed control of the temperature, operating pressures (inlet, outlet, and differential) and volume concentration factor during process. Once the process reached the target volume concentration factor, typically after 6-8 hours of operation, the retentate was diafiltered (DF) with one cubic meter of water, (approximately 5 parts of diafiltration water per part of concentrated retentate) to yield a high protein retentate. After a processing cycle, the system was cleaned with a typical CIP protocol used with most protein purification processes. The retentate contained about 80% dry basis protein after diafiltration.

The permeate of the UF/DF steps contained the sugars and was further concentrated in a Reverse Osmosis Membrane system (RO). The UF permeate was transferred to an RO system to concentrate the feed stream from approximately 2% total solids (TS) to 20% TS. The process equipment and membranes (RO98pHt) for the RO unit operation were supplied by Alfa-Laval. The feed pressure was increased in order to maintain a constant flux, up to 45 bar at a temperature of 50° C. Typically each batch started at a 2-3% Brix and end at 20-25% Brix (Brix=sugar concentration).

After the RO step the concentrated sugar stream was fed to an Electrodialysis Membrane (ED). Electrodialysis from Eurodia Industrie SA removes minerals from the sugar solution. The electrodialysis process has two product streams. One is the product, or diluate, stream which was further processed to concentrate and pasteurize the SOS concentrate solution. The other stream from the electrodialysis process is a brine solution which contains the minerals that were removed from the feed stream. The trial achieved >80% reduction in conductivity, resulting in a product stream that measured <3 mS/cm conductivity. The batch feed volume was approx 40 liters at a temperature of 40° C. and a pH of 7. The ED unit operated at 18V and had up to 50 cells as a stack size.

The de-mineralized sugar stream from the ED was further processed in an Evaporation step. The evaporation of the SOS stream was carried out on Anhydro's Lab E vacuum evaporator. SOS product was evaporated to 40-75% dry matter with a boiling temperature of approximately 50-55° C. and a ΔT of 5-20° C.

A Spray Dryer was used to dry UF/DF retentate suspension. The UF diafiltrate retentate, with a solids content of approximately 8%, was kept stirred in a tank. The suspension was then fed directly to the spray dryer where it was combined with heated air under pressure and then sprayed through a nozzle. The dryer removed the water from the suspension and generated a dry powder, which was collected in a bucket after it was separated from the air stream in a cyclone. The feed suspension was thermally treated at 150° C. for 9 seconds before it entered the spray dryer to kill the microbiological organisms. The spray dryer was a Production Minor from the company Niro/GEA. The dryer was set up with co-current flow and a two fluid nozzle. The drying conditions varied somewhat during the trial. Feed temperatures were about 80° C., nozzle pressure was about 4 bars, and inlet air temperatures was about 250° C.

### Example 6

### Capture of Bulk Soy Whey Protein Whey Pre-Treatment Process and Cross-Flow Filtration Membranes

Approximately 8000 lbs of aqueous soy whey (also referred to as raw whey) at 110° F. and 4.57 pH from an isolated soy protein extraction and isoelectric precipitation continuous process was fed to a reaction vessel where the pH was increased to 5.3 by the addition of 50% sodium hydroxide. The pH-adjusted raw whey was then fed to a second reaction vessel with a 10 minute average residence time in a continuous process where the temperature was increased to 190° F. by the direct injection of steam. The heated and pH-adjusted raw whey was then cooled to 90 degrees F. by passing through a plate and frame heat exchanger with chilled water as the cooling medium. The cooled raw whey was then fed into an Alfa Laval VNPX510 clarifying centrifuge where the suspended solids, predominantly insoluble large molecular weight proteins, were separated and discharged in the underfiow to waste and the clarified centrate proceeded to the next reaction vessel. The pH of the clarified centrate, or pre-treated whey protein, was adjusted to 8.0 using 12.5% sodium hydroxide and held for 10 minutes prior to being fed into an Alfa Laval VNPX510 clarifying centrifuge where the suspended solids, predominantly insoluble minerals, were separated and discharged in the underflow to waste. The clarified centrate proceeded to a surge tank prior to ultrafiltration. Ultrafiltration of the clarified centrate proceeded in a feed and bleed mode at 90° F. using 3.8″ diameter polyethersulfone spiral membranes, PW3838C, made by GE Osmonics, with a 10 kDa molecular weight cut-off. Ultrafiltration continued until a 60× concentration of the initial feed volume was accomplished, which required about 4.5 hrs. The retentate, 114 lbs at 4.5% total solids and 8.2 pH, was transferred to a reaction vessel where the pH was adjusted to 7.4 using 35% hydrochloric acid. The retentate was then heated to 305° F. for 9 seconds via direct steam injection prior to flash cooling to 140° F. in a vacuum chamber. The material was then homogenized by pumping through a homogenizing valve at 6000 psi inlet and 2500 outlet pressure prior to entering the spray drier through a nozzle and orifice combination in order to atomize the solution. The spray drier was operated at 538° F. inlet temperature and 197° F. outlet temperature, and consisted of a drying chamber, cyclone and bag house. The spray dried soy whey protein, a total of 4 lbs, was collected from the cyclone bottom discharge.

### Example 7

### Capture of Bulk Soy Whey Protein Using Expanded Bed Adsorption (EBA) Chromatography

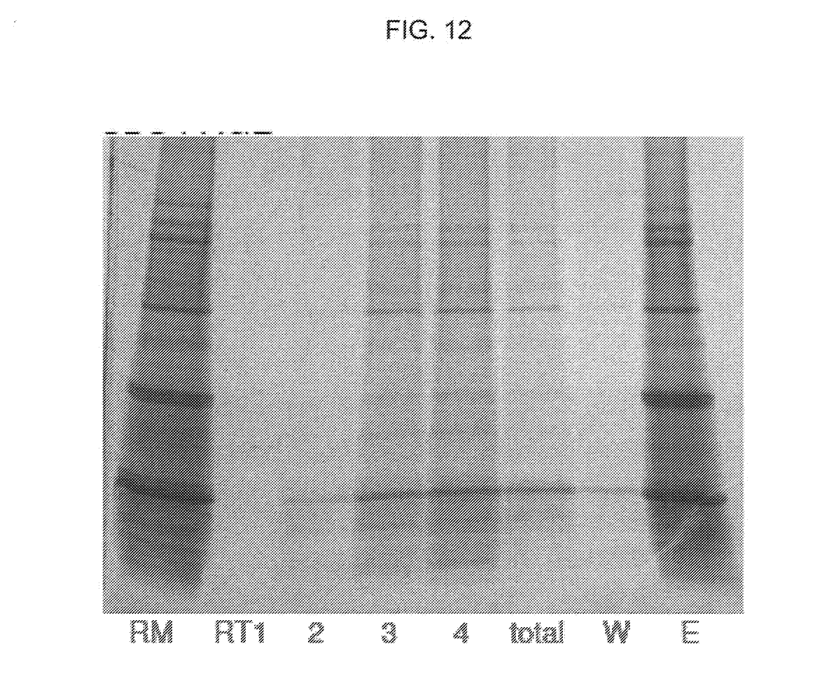

200 ml of aqueous raw soy whey (not pre-treated) with a total solids content of 1.92%, was adjusted to pH 4.5 with acetic acid and applied to a 1×25 cm column of Mimo6ME resin (UpFront Chromatography, Copenhagen Denmark) equilibrated in 10 mM sodium citrate, pH 4.5. Material was loaded onto the column from the bottom up at 20-25° C. using a linear flow rate of 7.5 cm/min. Samples of the column flow-through were collected at regular intervals for later analysis. Unbound material was washed free of the column with 10 column volumes of equilibration buffer, then bound material recovered by elution with 50 mM sodium hydroxide. 10 μls of each fraction recovered during EBA chromatography of aqueous soy whey were separated on a 4-12% SDS-PAGE gel and stained with Coomassie Brilliant Blue R 250 stain. SDS-PAGE analysis of the column load, flow-through, wash, and sodium hydroxide eluate samples is depicted in FIG. 12. As used in FIG. 12, RM: raw material (column load); RT1-4: column flow-through (run through) collected at equal intervals during the load; total: the total run-through fraction; W: column wash; E: column eluate. Binding was reasonably efficient, as very little protein is seen in the initial breakthrough fractions, only showing up in the later fractions. A total of 662 mg of protein was recovered in the eluate, for a yield of 3.3 mg/ml of starting material. Under these conditions, the capacity of this resin was shown to be 33.1 mg of protein per ml of adsorbant.

### Example 8

### Capture of Bulk Soy Whey Protein from Spray-Dried SWP Using Expanded Bed Adsorption (EBA) Chromatography

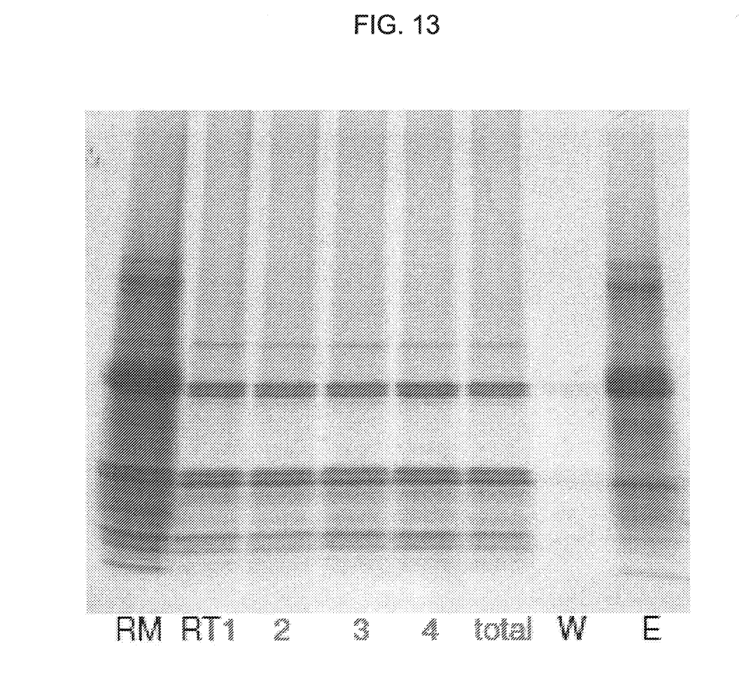

Spray-dried soy whey powder was slurried to a concentration of 10 mg/ml in water and adjusted to pH 4.0 with acetic acid. 400 ml of the slurry was then applied directly to the bottom of a 1×25 cm column of Mimo-4SE resin (UpFront Chromatography, Copenhagen Denmark) that had been equilibrated in 10 mM sodium citrate, pH 4.0. Material was loaded at 20-25° C. using a linear flow rate of 7.5 cm/min. Samples of the column flow-through were collected at regular intervals for later analysis. Unbound material was washed free of the column using 10 column volumes of equilibration buffer. Bound material was eluted with 30 mM NaOH. 10 μls of each fraction recovered during EBA chromatography of a suspension of soy whey powder were separated on a 4-12% SDS-PAGE gel and stained with Coomassie Brilliant Blue R 250 stain. SDS-PAGE analysis of the column load, flow-thru, wash, and eluate are depicted in FIG. 13. As used in FIG. 13, RM: raw material (column load); RT1-4: column flow-through (run through) collected at equal intervals during the load; total: the total run-through fraction; W: column wash; E: column eluate. Binding was not as efficient as was observed using the Mimo6ME resin, as several protein bands are seen in the breakthrough fractions. A total of 2070 mg of protein were recovered in the eluate, for a yield of 5.2 mg/ml of starting material. Under these conditions, the capacity of this resin was shown to be 104 mg of protein per ml of adsorbant.

### Example 9

### Removal of KTI from Bulk Soy Whey Protein Using Expanded Bed Adsorption (EBA) Chromatography

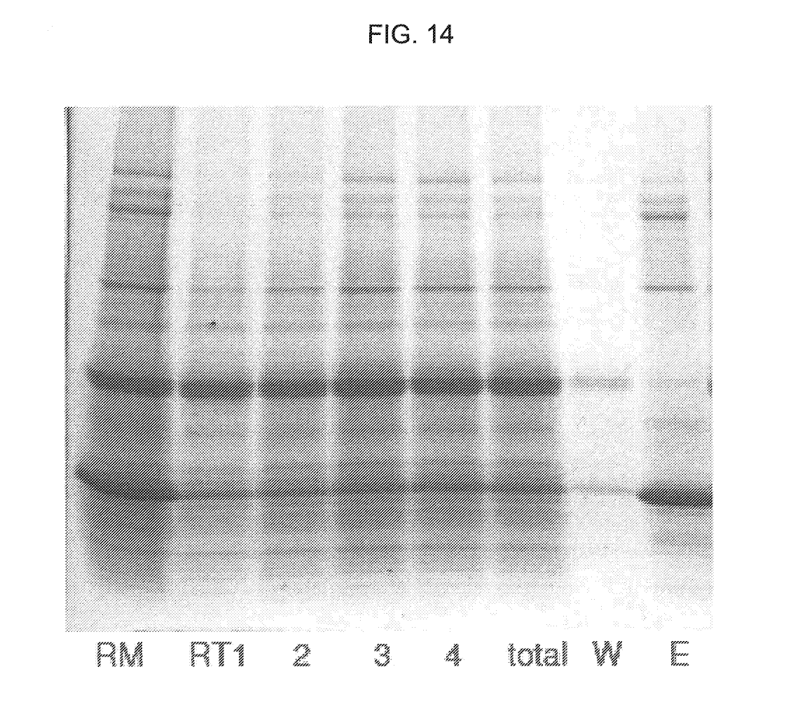

Two procedures were used to remove the majority of contaminating KTI protein from the bulk of the soy whey protein by EBA chromatography. In the first, 200 ml of aqueous raw soy whey (not pre-treated) with a total solids content of 1.92%, was adjusted to pH 6.0 with sodium hydroxide and applied to a 1×25 cm column of Mimo6HE resin (UpFront Chromatography, Copenhagen Denmark) equilibrated in 10 mM sodium citrate, pH 6.0. Material was loaded onto the column from the bottom up at 20-25° C. using a linear flow rate of 7.5 cm/min. Samples of column flow-through were collected at regular intervals for later analysis. Unbound material was washed free of the column with 10 column volumes of equilibration buffer, then bound material recovered by elution with 30 mM sodium hydroxide. 10 μls of each fraction recovered during EBA chromatography of a suspension of soy whey powder were separated on a 4-12% SDS-PAGE gel and stained with Coomassie Brilliant Blue R 250 stain. SDS-PAGE analysis of the column load, flow-through, wash, and sodium hydroxide eluate samples is depicted in FIG. 14. As used in FIG. 14, RM: raw material (column load); RT1-4: flow-through material (run through) collected at equal intervals during the load; total: the total run-through fraction; W: column wash; E: column eluate. The bulk of the loaded protein is clearly seen eluting in the flow-through, while the bulk of the KTI protein remains bound to the resin. A total of 355 mg of protein, the bulk of which is KTI, was recovered in the eluate, for a yield of 1.8 mg/ml of starting material. Under these conditions, the capacity of this resin was shown to be 17.8 mg of KTI (plus minor contaminants) per ml of adsorbant.

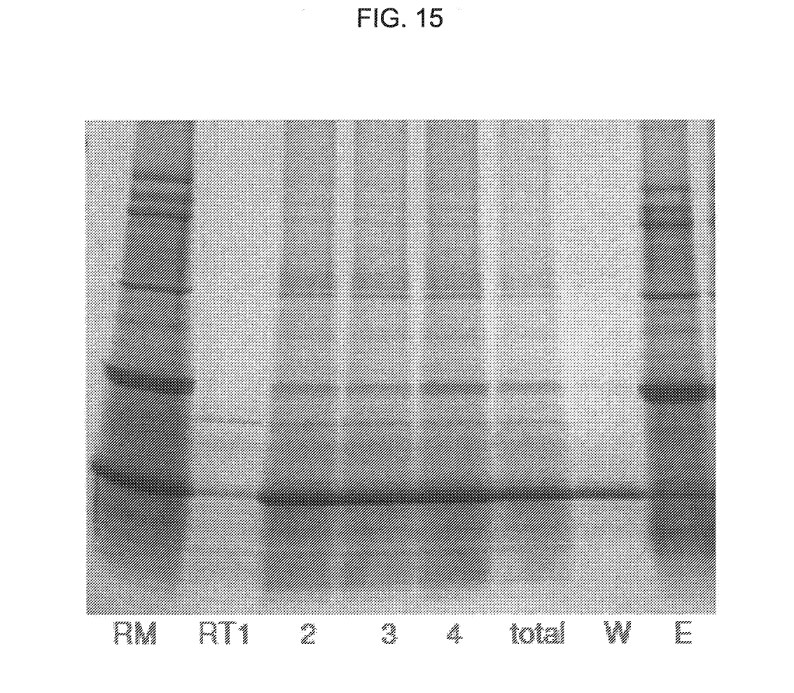

In the second procedure, 160 mls of aqueous raw soy whey (not pre-treated) with a total solids content of 1.92%, was adjusted to pH 5.1 with acetic acid and applied to a 1×25 cm column of Mimo6ZE resin (UpFront Chromatography, Copenhagen Denmark) equilibrated in 10 mM sodium citrate, pH 5.0. Material was loaded onto the column from the bottom up at 20-25° C. using a linear flow rate of 7.5 cm/min. Samples of column flow-through were collected at regular intervals for later analysis. Unbound material was washed free of the column with 10 column volumes of equilibration buffer, then bound material recovered by elution with 30 mM sodium hydroxide. 10 μls of each fraction recovered during EBA chromatography of a suspension of soy whey powder were separated on a 4-12% SDS-PAGE gel and stained with Coomassie Brilliant Blue R 250 stain. SDS-PAGE analysis of the column load, flow-through, wash, and sodium hydroxide eluate samples is depicted in FIG. 15. As used in FIG. 15, RM: raw material (column load); RT1-4: flow-through material (run through) collected at equal intervals during the load; total: the total run-through fraction; W: column wash; E: column eluate. The bulk of the KTI is clearly seen eluting in the flow-through, while the bulk of the remaining protein remains bound to the resin. A total of 355 mg of soy protein essentially devoid of contaminating KTI was recovered in the eluate, for a yield of 2.1 mg/ml of starting material. Under these conditions, the capacity of this resin was shown to be 16.8 mg of soy protein per ml of adsorbant.

One skilled in the art would readily appreciate that the methods and compositions described herein are representative of exemplary embodiments, and not intended as limitations on the scope of the invention. It will be readily apparent to one skilled in the art that varying substitutions and modifications may be made to the present disclosure disclosed herein without departing from the scope and spirit of the invention.

All patents and publications mentioned in the specification are indicative of the levels of those skilled in the art to which the present disclosure pertains. All patents and publications are herein incorporated by reference to the same extent as if each individual publication was specifically and individually indicated as incorporated by reference.

The present disclosure illustratively described herein suitably may be practiced in the absence of any element or elements, limitation or limitations that are not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the present disclosure claimed. Thus, it should be understood that although the present disclosure has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method for recovering soy whey protein from a processing stream, wherein the process comprises performing the following steps in any order:
(a) pretreating the feed stream by passing the stream through at least one separation technique to form a stream comprised of soluble components of the soy whey in the aqueous phase and a stream comprised of insoluble large molecular weight proteins, wherein the insoluble large molecular weight proteins are storage proteins;
(b) passing the pre-treated soy whey through to at least one separation technique to form a stream comprised of various components including but not limited to storage proteins, microorganisms, silicon, and combinations thereof, and a stream comprised of purified pre-treated soy whey;
(c) passing the purified pre-treated soy whey stream of (b) through at least one separation technique to form a stream comprised of purified pre-treated soy whey and a stream comprised of water, some minerals, monovalent cations, and combinations thereof;
(d) passing the purified pre-treated soy whey stream of (c) through at least one separation technique to form a suspension of purified pre-treated soy whey and precipitated minerals;
(e) passing the suspension of purified pre-treated soy whey and precipitated minerals of (d) through at least one separation technique to form a stream comprised of de-mineralized pre-treated soy whey and a stream comprised of insoluble materials with protein mineral complexes;
(f) passing the de-mineralized purified pre-treated soy whey stream of (e) through at least one separation technique to form a stream comprised of proteins selected from the group consisting of soy whey protein, BBI, KTI, storage proteins, other proteins and combinations thereof and a stream comprised of peptides, soy oligosaccharides, minerals, and combinations thereof;
(g) passing the proteins of (f) through at least one separation technique to form a stream comprised of proteins selected from the group consisting of soy whey protein, BBI, KTI, storage proteins, other proteins, and combinations thereof and a stream comprised of peptides, water, minerals, and soy oligosaccharides, wherein the soy oligosaccharides are selected from the group consisting of sucrose, raffinose, stachyose, verbascose, monosaccharides, and combinations thereof;
(h) passing the proteins of (g) through at least one separation technique to form a stream comprising peptides, soy oligosaccharides, water, minerals and a stream comprising water and minerals;
(i) passing the stream of (h) through at least one separation technique to form a stream comprising de-mineralized soy oligosaccharides and a stream comprising minerals, water and combinations thereof;
(j) passing the de-mineralized soy oligosaccharides (i) through at least one separation technique to form a stream comprising color compounds and a stream comprising soy oligosaccharides;
(k) passing the soy oligosaccharides of (j) through at least one separation technique to form a stream comprising sucrose, monosaccharides, and combinations thereof and a stream comprising raffinose, stachyose, verbascose and combinations thereof;
(l) passing the stream of (k) through at least one separation technique to form a stream comprising water and a stream comprising soy oligosaccharides;
(m) passing the stream of (g) through at least one separation technique to form a stream comprising soy oligosaccharides, water, and minerals and a stream comprising peptides and other proteins;
(n) passing the stream of (m) through at least one separation technique to form a stream comprising water and a stream comprising peptides and other proteins, wherein the proteins are selected from the group consisting of lunasin, lectins, dehydrins, lipoxygenase, and combinations thereof;
(o) passing the stream of (f) through at least one separation technique to form a stream comprising storage proteins and a stream comprising soy whey protein, BBI, KTI, and other proteins, wherein the other proteins are selected from the group consisting of lunasin, lectins, dehydrins, lipoxygenase and combinations thereof;
(p) passing the stream of (o) through at least one separation technique to form a stream comprising water and a stream comprising soy whey protein, BBI, KTI and other proteins; and
(q) heating, flash cooling and drying the stream of (p) to form soy whey protein.

2. A soy whey protein composition comprising soy proteins having a solubility of at least about 80% in an acidic aqueous medium across a pH range of the aqueous medium of from about 2 to about 10 and a temperature of about 25° C.

3. The soy whey protein composition of claim 2, wherein the composition is derived from a soy processing stream.

4. The soy protein composition of claim 2 wherein the pH of the acidic aqueous medium is from about 2 to about 7.

5. The soy protein composition of claim 2, wherein the composition has a viscosity of from about 2 to 10 cP.

6. The soy protein composition of claim 2, wherein the composition has a viscosity of from about 3.6 to 7.5 cP.

7. A soy whey protein composition comprising soy proteins having a solubility of at least about 80% in an acidic aqueous medium across a pH range of the aqueous medium of from about 2 to about 10 and a temperature of about 25° C., and a viscosity of from about 2 to 10 cP, wherein the composition is derived from a soy processing stream.
